(19) United States
(12) Patent Application Publication
(10) Pub. No.: US 2022/0401541 A1
(43) Pub. Date: Dec. 22, 2022

(54) INTRATUMORAL ADMINISTRATION OF IMMUNE CELLULAR THERAPEUTICS

(71) Applicant: FIGENE, LLC, Houston, TX (US)

(72) Inventors: Thomas ICHIM, San Diego, CA (US); Pete O'HEERON, Houston, TX (US)

(21) Appl. No.: 17/755,278

(22) PCT Filed: Nov. 2, 2020
(86) PCT No.: PCT/US2020/058497

§ 371 Date: Apr. 26, 2022

Related U.S. Application Data
(60) Provisional application No. 62/929,830, filed on Nov. 2, 2019.

(51) Int. Cl. A61K 39/00; A61K 39/39; A61K 31/675; A61K 45/06

(52) U.S. Cl. CPC A61K 39/001195; A61K 39/001109; A61K 39/001108; A61K 39/001186; A61K 39/001188; A61K 39/001166; A61K 39/00117; A61K 39/001171; A61K 39/001106; A61K 39/001168; A61K 39/39; A61K 31/675; A61K 45/06; A61K 2039/5156; A61K 2039/55516; A61K 2039/55561; A61K 2039/55588; A61K 2039/55572; A61K 2039/5154; A61K 2039/55533; A61K 2039/55527

(57) ABSTRACT

Embodiments of the disclosure include methods and compositions useful for treating cancer in an immunogenic manner so as to elicit local tumor regression, while priming systemic immunity. In one embodiment, there is expansion of tumor-specific immune cells through administration of fibroblasts, either natural or modified in an intratumoral and/or peritumoral manner. In other embodiments, manipulation of a local tumor microenvironment is achieved by injections of immune-modulating fibroblasts to facilitate expansion of immune effector cells, which are subsequently re-stimulated in the periphery by antigenic exposure. In another embodiment, agents are provided that allow for systemic derepression of immunity, while optionally augmenting ability of immune effector cells to expand and kill tumor cells.

This application claims priority to U.S. Provisional Patent Application No. 62/929,830, filed Nov. 2, 2019, which is incorporated by reference herein in its entirety.

### TECHNICAL FIELD

Embodiments of the field of the disclosure concern at least the fields of cell biology, molecular biology, immunology, and medicine, including cancer medicine.

### BACKGROUND

Cancer immunotherapy began at the turn of the 20^{th} Century with the work of William Coley, who was able to successfully induce tumor regression by administration of a mixture consisting of killed bacteria of species Streptococcus pyogenes and Serratia marcescens [1-8]. In more recent years, the FDA approval of the Provenge dendritic cell vaccine for prostate cancer (2010) [9], Ipilimumab (Yervoy) anti-CTLA4 antibody for treatment of melanoma (2011) [10], Pembrolizumab (Keytruda) anti-PD1 antibody for melanoma (2014) [11], and Nivolumab (Opdivo) for melanoma (2014) [12], ushered a new age of immunotherapy. Despite improved survival using these novel immune modulators, not all patients respond, with an average of 20% remissions being reported [13].

The embodiments of the disclosure address multiple aspects of the immune system in order to augment possibility of increasing overall survival. Specifically, it is known from studies of immune modulators that recruitment of multiple arms of the immune system associates with increased efficacy. For example, it is known that natural killer (NK) cells play an important role in immune destruction of cancer [14-20]. A clinical trial demonstrated that patients who possess elevated levels of natural killer cell inhibitory proteins (soluble NKG2D ligands) demonstrated lower responses to checkpoint inhibitors [21]. Indeed this should not be surprising because studies show that NK cell infiltration of tumors induces upregulation of antigen presentation in an interferon gamma associated manner, which renders tumor cells sensitive to T cell killing [22]. Another example of the potency of combining immunotherapies is the example of Herceptin, in which approximately 1 out of 4 patients with the HER2neu antigen respond to treating. Interestingly it was found that lack of responsiveness correlates with inhibited NK cell activity [23-25]. Indeed, animal experiments demonstrate augmentation of Herceptin activity by stimulators of NK cells such as Poly (IC) and interleukin (IL)-12 [26, 27]. The current disclosure provides solutions to integrate the main arms of the immune system so as to achieve a synergistic induction of anticancer immunity.

### BRIEF SUMMARY

The present disclosure is directed to methods and compositions that concern cell therapy and immunotherapy for treating an individual in need of therapy, including an individual in need of therapy for cancer of any kind, including solid tumors or hematological malignancies, for example. In particular cases, the individual is a mammal, including a human, dog, cat, horse, and so forth. The individual may have a disease or medical condition for which the cell therapy is effective, including for amelioration of at least one symptom. The disclosure also includes methods of preventing any disease or medical condition, such as for an individual having an elevated risk for the disease or medical condition compared to another (for example, having a personal or family history, having a genetic marker associated with the disease or medical condition, being a smoker and/or obese, and so forth) or an individual being suspected of having the disease or medical condition. The methods and compositions relate to any disease or medical condition having at least one associated cell antigen to which an antibody of any kind may target. The therapy may remove the symptom, reduce the severity of the symptom, and/or delay the onset of the symptom. In specific embodiments, the individual has cancer or is at risk for having cancer (e.g., an elevated risk compared to the general population) or is susceptible to having cancer.

In particular embodiments, the disclosure concerns the administration of fibroblasts in combination with immunogenic and immune stimulatory compositions and a composition or action capable of inducing cancer cell death, where the administration is to an individual having, or at risk of having, a tumor or non-tumorous cancer. The fibroblasts may reduce cancer-associated immune suppression, including immune suppression that causes immune cells in the individual not to attack and destroy the cancer cells, including destroy the tumor. Immunogenic compositions disclosed herein may induce an immune reaction against one or more antigens present on a cancer cell in an individual. Inducing such an immune reaction can immunize the individual against the cancer. Immune stimulatory compositions may increase the presentation of one or more antigens present on a cancer cell in an individual and/or increase the response of the immune system against antigens on the cancer cells, including the antigens immunized by the immunogenic composition.

Fibroblasts, as present in aspects of the disclosure, are capable of reducing tumor-associated immune suppression. The fibroblasts possess anti-inflammatory activity useful in embodiments of the present disclosure. The anti-inflammatory activity may be of any kind including suppressing the production of TNF-alpha, IL-1, IL-6, or a combination thereof in cells endogenous to the individual that are cancerous or at risk for becoming cancerous. The fibroblasts may be manipulated to possess activities and capabilities useful for methods of the present disclosure, including the capability to reduce tumor-associated immune suppression and/or anti-inflammatory activity. In some embodiments, the fibroblasts are manipulated in culture by exposure to one or more compositions including, but not limited to, IL-10, indomethacin, valproic acid, naltrexone (including low dose naltrexone), IL-27, or a combination thereof.

In specific embodiments, the methods of the disclosure encompasses immunizing a patient against a tumor antigen and then providing an effective amount of fibroblasts after immunization. Thus, in specific embodiments the tumors are sensitized with fibroblasts that have anti-inflammatory activity. In specific methods, fibroblasts are given to the individual followed by immunization, followed by giving fibroblasts again after immunizing the individual.

Fibroblasts disclosed herein, may express one or more certain surface markers, including, but not limited to, CD117, CD105, Oct-4, CD-34, KLF-4, Nanog, Sox-2, Rex-1, GDF-3, Stella, GDF-11, or a combination thereof. The fibroblasts may express flu peptides, such as peptides derived from the influenza virus. The fibroblasts may express markers that are useful for purifying the fibroblasts. The fibroblasts may also express other markers that are useful for the methods disclosed herein.

Immunogenic compositions of the present disclosure may comprise a vaccine, a peptide, plurality of peptides, peptide mimic, or other composition that induces an immune response. In some embodiments, the immunogenic composition may induce or cause the expansion of any type of immune cells, including immune cells that can target and/or destroy a tumor. The immunogenic composition may comprise one or more antigens that are expressed on cancer cells, present in the microenvironment of a tumor, or otherwise be associated with a tumor. The antigen may be at least one of Fos-related antigen 1, LCK, FAP, VEGFR2, NA17, PDGFR-beta, PAP, MAD-CT-2, Tie-2, PSA, protamine 2, legumain, endosialin, prostate stem cell antigen, carbonic anhydrase IX, STn, Page4, proteinase 3, GM3 ganglioside, tyrosinase, MART1, gp100, SART3, RGS5, SSX2, Globol1, Tn, CEA, hCG, PRAME, XAGE-1, AKAP-4, TRP-2, B7H3, sperm fibrous sheath protein, CYP1B1, HMWMAA, sLe(a), MAGE A1, GD2, PSMA, mesothelin, fucosyl GM1, GD3, sperm protein 17, NY-ESO-1, PAX5, AFP, polysialic acid, EpCAM, MAGE-A3, mutant p53, ras, mutant ras, NY-BR1, PAX3, HER2/neu, OY-TES1, HPV E6 E7, PLAC1, hTERT, BORIS, ML-IAP, idiotype of b cell lymphoma or multiple myeloma, EphA2, EGFRvIII, cyclin B1, RhoC, androgen receptor, surviving, MYCN, wildtype p53, LMP2, ETV6-AML, MUC1, BCR-ABL, ALK, WT1, ERG (TMPRSS2 ETS fusion gene), sarcoma translocation breakpoint, STEAP, OFA/iLRP, Chondroitin sulfate proteoglycan 4 (CSPG4), Epithelial tumor antigen, alphafetoprotein, CD19, CA-125, or a combination thereof.

The immunogenic composition may be derived from a cancer cell or tumor, including a tumor afflicting an individual in need of methods and compositions of the present disclosure. The immunogenic composition may comprise lysate from a tumor, mRNA extracted from tumor, exosomes from a tumor, or otherwise be derived from a tumor or part thereof. The lysate, mRNA, exosomes, or otherwise may be used to induce the expression or abundance of an antigen, including antigens of the present disclosure, in a cell, such as an antigen-presenting cell (including fibroblasts) or any other cell useful for expressing antigens. The immunogenic composition may or may not be matched to the HLA of an individual, including individuals in need of methods and compositions of the present disclosure.

In certain embodiments, an adjuvant is administered with the compositions of the present disclosure. The adjuvant may stimulate antigen presentation. In some embodiments the adjuvant comprises a toll-like receptor, including TLR-2, TLR-3, TLR-4, TLR-5, TLR-7, TLR-8, TLR-9, or a combination thereof. The adjuvant comprising TLR-2 may be activated by any activator of TLR-2 including Pam3cys4, heat killed Listeria monocytogenes (HKLM), FSL-1, or a combination thereof. The adjuvant comprising TLR-3 may be activated by any activator of TLR-3 including Poly IC, double stranded RNA, or both. The double stranded RNA may be of any origin, including mammalian and/or bacterial. The double stranded RNA may comprise leukocyte extract, such as leukocyte extract from freeze-thawed leukocytes. The freeze thawed leukocytes may be dialyzed for compounds less than 15 kDa. The adjuvant comprising TLR-4 may be activated by any activator of TLR-4 including lipopolysaccharide, HMGB-1 (including peptides from HMGB-1, such as hp91 for example), or a combination thereof. The adjuvant comprising TLR-4 may be activated by a peptide comprising at least 80, 85, 90, 95, 96, 97, 98, 99, or 100 percent identity to the peptide with an amino acid sequence of EFDVILKAAGANKVAVIKAVRGATGLGLKEAKDLVESAPAALKEGVSKDDAEALKKAL EEAGAEVEVK (SEQ ID NO:1). The adjuvant comprising TLR-5 may be activated by any activator of TLR-5 including flagellin, The adjuvant comprising TLR-7 may be activated by any activator of TLR-7 including imiquimod. The adjuvant comprising TLR-8 may be activated by any activator of TLR-8 including resmiquimod. The adjuvant comprising TLR-9 may be activated by any activator of TLR-9 including CpG DNA.

In certain embodiments, the adjuvant that stimulates antigen presentation may increase the expression of at least one costimulatory molecule on antigen presenting cells. The costimulatory molecule may be any molecule, including CD40, CD80, CD86, or a combination thereof. The adjuvant increasing the expression of at least one costimulatory molecules may comprise an activator of NF-kappa-B, including an inhibitor of i-kappa-B, an activator of a PAMP receptor, or other NF-kappa-B activators. The PAMP receptor may be any PAMP receptor including MDA5, RIG-1, NOD, or a combination thereof. The adjuvant increasing the expression of at least one costimulatory molecule may comprise an activator of the JAK-STAT pathway. The JAK-STAT activator may be any activator of the JAK-STAT pathway, including interferon gamma.

In some embodiments, the immune stimulatory composition may be capable of augmenting antigen presentation. The composition capable of augmenting antigen presentation may be a dendritic cell, including an activated dendritic cell. The dendritic cell may be activated by one or more TLR agonists, one or more PAMP agonists, or a combination thereof. The dendritic cell may be activated by in vivo administration of GM-CSF, FLT-3L or a combination thereof. The dendritic cell may be generated from a different cell, such a monocyte. Any cells, including dendritic cells may be autologous or allogenic with respect to an individual subjected to methods and compositions of the present disclosure.

Particular embodiments of the present disclosure concern a composition or action that induces cancer cell death. The composition or action may be any composition or action known in the art to induce cancer cell death. The action that induces cancer cell death may be the administration of localized radiation therapy and/or hyperthermia. The action that induces cancer cell death may be cryoablation therapy to the cancer. The composition that induces cancer cell death may be comprise a chemotherapy, including but not limited to, acivicin, aclarubicin, acodazole hydrochloride, acronine, adozelesin, aldesleukin, altretamine, ambomycin, ametantrone acetate, aminoglutethimide, amsacrine, anastrozole, anthramycin, asparaginase, asperlin, azacitidine, azetepa, azotomycin, batimastat, benzodepa, bicalutamide, bisantrene hydrochloride, bisnafide dimesylate, bizelesin, bleomycin sulfate, brequinar sodium, bropirimine, busulfan, cactinomycin, calusterone, caracemide, carbetimer, carboplatin, carmustine, carubicin hydrochloride, carzelesin, cedefingol, chlorambucil, cirolemycin, cisplatin, cladribine, crisnatol mesylate, cyclophosphamide, cytarabine, dacarbazine, dactinomycin, daunorubicin hydrochloride, decitabine, dexormaplatin, dezaguanine, dezaguanine mesylate, diaziquone, docetaxel, doxorubicin, doxorubicin hydrochloride, droloxifene, droloxifene citrate, dromostanolone propionate, duazomycin, edatrexate, eflornithine hydrochloride, elsamitrucin, enloplatin, enpromate, epipropidine, epirubicin hydrochloride, erbulozole, esorubicin hydrochloride, estramustine, estramustine phosphate sodium, etanidazole, etoposide, etoposide phosphate, etoprine, fadrozole hydrochloride, fazarabine, fenretinide, floxuridine, fludarabine phosphate, fluorouracil, fluorocitabine, fosquidone, fostriecin sodium, gemcitabine, gemcitabine hydrochloride, hydroxyurea, idarubicin hydrochloride, ifosfamide, ilmofosine, interleukin II (including recombinant interleukin II, or rIL2), interferon alfa-2a, interferon alfa-2b, interferon alfa-n1, interferon alfa-n3, interferon beta-I a, interferon gamma-I b, iproplatin, irinotecan hydrochloride, lanreotide acetate, letrozole, leuprolide acetate, liarozole hydrochloride, lometrexol sodium, lomustine, losoxantrone hydrochloride, masoprocol, maytansine, mechlorethamine hydrochloride, megestrol acetate, melengestrol acetate, melphalan, menogaril, mercaptopurine, methotrexate, methotrexate sodium, metoprine, meturedepa, mitindomide, mitocarcin, mitocromin, mitomalcin, mitomycin, mitosper, mitotane, mitoxantrone hydrochloride, mycophenolic acid, nocodazole, nogalamycin, ormaplatin, oxisuran, paclitaxel, pegaspargase, peliomycin, pentamustine, peplomycin sulfate, perfosfamide, pipobroman, piposulfan, piroxantrone hydrochloride, plicamycin, plomestane, porfimer sodium, porfiromycin, prednimustine, procarbazine hydrochloride, puromycin, puromycin hydrochloride, pyrazofurin, riboprine, rogletimide, safingol, safingol hydrochloride, semustine, simtrazene, sparfosate sodium, sparsomycin, spirogermanium hydrochloride, spiromustine, spiroplatin, streptonigrin, streptozocin, sulofenur, talisomycin, tecogalan sodium, tegafur, teloxantrone hydrochloride, temoporfin, teniposide, teroxirone, testolactone, thiamiprine, thioguanine, thiotepa, tiazofurin, tirapazamine, toremifene citrate, trestolone acetate, triciribine phosphate, trimetrexate, trimetrexate glucuronate, triptorelin, tubulozole hydrochloride, uracil mustard, uredepa, vapreotide, verteporfin, vinblastine sulfate, vincristine sulfate, vindesine, vindesine sulfate, vinepidine sulfate, vinglycinate sulfate, vinleurosine sulfate, vinorelbine tartrate, vinrosidine sulfate, vinzolidine sulfate, vorozole, zeniplatin, zinostatin, zorubicin hydrochloride, or a combination thereof.

In some embodiments of the present disclosure, prior to the administration of compositions or action disclosed herein, a state of lymphopenia may be induced in an individual of the present disclosure. The lymphopenia may induce homeostatic expansion of lymphocytes in the individual, which may reduce the need for co-stimulatory molecules, such as a reduction in the need for co-stimulatory molecules by approximately 10%, 20%, 30%, 40%, 50%, 60%, 70%, 80%, 90%, 100%, or more. The lymphopenia may be induced by irradiation (e.g. total lymphoid irradiation), cyclophosphamide, or both.

In some embodiments, an immune-depressing composition is administered to an individual of the present disclosure. The immune-depressing composition may be a phosphodiesterase (PDE)-5 inhibitor, such as acetildenafi, aildenafil, avanafil, benzamidenafil, homosildenafil, icariin, lodenafil, mirodenafil, nitrosoprodenafil, sildenafil, sulfoaildenafil, tadalafil, udenafil, vardenafil, zaprinast, or a combination thereof.

The individual of the present disclosure may have any type of cancer, including a brain tumor. The brain tumor may be, for example, a glioblastoma, a glioblastoma multiforme, an oligodendroglioma, a primitive neuroectodermal tumor, an astrocytoma, an ependymoma, an oligodendroglioma, a medulloblastoma, a meningioma, a pituitary carcinoma, a neuroblastoma, a craniopharyngioma, or a combination thereof.

The foregoing has outlined rather broadly the features and technical advantages of the present disclosure in order that the detailed description that follows may be better understood. Additional features and advantages will be described hereinafter which form the subject of the claims herein. It should be appreciated by those skilled in the art that the conception and specific embodiments disclosed may be readily utilized as a basis for modifying or designing other structures for carrying out the same purposes of the present designs. It should also be realized by those skilled in the art that such equivalent constructions do not depart from the spirit and scope as set forth in the appended claims. The novel features which are believed to be characteristic of the designs disclosed herein, both as to the organization and method of operation, together with further objects and advantages will be better understood from the following description. It is to be expressly understood, however, that the description is provided for the purpose of illustration only and is not intended as a definition of the limits of the present disclosure.

### DETAILED DESCRIPTION

### Definitions

Unless defined differently, all technical and scientific terms used herein have the same meanings as commonly understood by one of skill in the art to which the disclosed disclosure belongs. In particular, the following terms and phrases have the following meaning.

The term, “adjuvant” refers to a substance that is capable of enhancing, accelerating, or prolonging an immune response when given with a vaccine immunogen or any immunogenic composition.

As used herein, the term “agonist” refers to a substance that promotes (induces, causes, enhances or increases) the activity of another molecule or a receptor. The term agonist encompasses substances which bind receptors (e.g., an antibody, a homolog of a natural ligand from another species) and substances which promote receptor function without binding thereto (e.g., by activating an associated protein).

The term “antagonist” or “inhibitor” refers to a substance that partially or fully blocks, inhibits, or neutralizes a biological activity of another molecule or receptor. The antagonist or inhibitor may be a protein or small molecule, and may be an antibody, for example.

“Co-administration” refers to administration of two or more agents to the same subject during a treatment period. The two or more agents may be encompassed in a single formulation and thus be administered simultaneously. Alternatively, the two or more agents may be in separate physical formulations and administered separately, either sequentially or simultaneously to the subject. The term “administered simultaneously” or “simultaneous administration” means that the administration of the first agent and that of a second agent overlap in time with each other, while the term “administered sequentially” or “sequential administration” means that the administration of the first agent and that of a second agent does not overlap in time with each other.

“Immune response” refers to any detectable response to a particular substance (such as an antigen or immunogen) by the immune system of a host vertebrate animal, including, but not limited to, innate immune responses (e.g., activation of Toll receptor signaling cascade), cell-mediated immune responses (e.g., responses mediated by T cells, such as antigen-specific T cells, and non-specific cells of the immune system), and humoral immune responses (e.g., responses mediated by B cells, such as generation and secretion of antibodies into the plasma, lymph, and/or tissue fluids). Examples of immune responses include an alteration (e.g., increase) in Toll-like receptor activation, lymphokine (e.g., cytokine (e.g., Th1, Th2 or Th17 type cytokines) or chemokine) expression or secretion, macrophage activation, dendritic cell activation, T cell (e.g., CD4+ or CD8+ T cell) activation, NK cell activation, B cell activation (e.g., antibody generation and/or secretion), binding of an immunogen (e.g., antigen (e.g., immunogenic polypolypeptide)) to an MHC molecule, induction of a cytotoxic T lymphocyte (“CTL”) response, induction of a B cell response (e.g., antibody production), and, expansion (e.g., growth of a population of cells) of cells of the immune system (e.g., T cells and B cells), and increased processing and presentation of antigen by antigen presenting cells. The term “immune response” also encompasses any detectable response to a particular substance (such as an antigen or immunogen) by one or more components of the immune system of a vertebrate animal in vitro.

As used herein, “immunization” refers to inducing an immune response in an individual against at least one specific molecule. Immunization may be carried out by administering an immunogenic composition, including any immunogenic composition disclosed herein. “Immunity” refers to an individual, having been immunized or otherwise, capable of inducing an immune response against at least one specific molecule, including specific molecules, such as antigens, disclosed herein.

“Immunogenic fibroblasts” are fibroblasts that elicit an immune response upon administration. Such fibroblasts include naturally immunogenic fibroblasts such as allogeneic or xenogeneic fibroblasts, or fibroblasts that have been cultured to endow immunogenicity. Alternatively, immunogenic fibroblasts comprise fibroblasts transfected with tumor antigens or other antigens capable of stimulating immunity. Antigens include tumor antigens, influenza antigens, antigens to which a pre-existing immunity is present in the patient, and antigens capable of augmenting immunity.

“Treating a cancer”, “inhibiting cancer”, “reducing cancer growth” refers to inhibiting or preventing oncogenic activity of cancer cells. Oncogenic activity can comprise inhibiting migration, invasion, drug resistance, cell survival, anchorage-independent growth, non-responsiveness to cell death signals, angiogenesis, or combinations thereof of the cancer cells. The terms “cancer”, “cancer cell”, “tumor”, and “tumor cell” are used interchangeably herein and refer generally to a group of diseases characterized by uncontrolled, abnormal growth of cells (e.g., a neoplasioa). In some forms of cancer, the cancer cells can spread locally or through the bloodstream and lymphatic system to other parts of the body (“metastatic cancer”). The treatment may delay onset of the cancer, reduce the severity of at least one symptom of cancer, delay metastasis of the cancer, reduce the severity of the metastasis of the cancer, and so forth.

“Ex vivo activated lymphocytes”, “lymphocytes with enhanced antitumor activity” and “dendritic cell cytokine induced killers” are terms used interchangeably to refer to composition of cells that have been activated ex vivo and subsequently reintroduced within the context of the current disclosure. Although the word “lymphocyte” is used, this also includes heterogenous cells that have been expanded during the ex vivo culturing process including dendritic cells, NKT cells, gamma delta T cells, and various other innate and adaptive immune cells.

As used herein, “cancer” refers to all types of cancer or neoplasm or malignant tumors found in animals, including leukemias, carcinomas and sarcomas. Examples of cancers are cancer of the brain, melanoma, bladder, breast, cervix, colon, head and neck, kidney, lung, non-small cell lung, mesothelioma, ovary, prostate, sarcoma, stomach, spleen, endometrium, thyroid, gall bladder, blood, pancreas, uterus and Medulloblastoma. In some particular embodiments of the disclosure, the cancer treated is a melanoma.

The term “leukemia” is meant broadly progressive, malignant diseases of the hematopoietic organs/systems and is generally characterized by a distorted proliferation and development of leukocytes and their precursors in the blood and bone marrow. Leukemia diseases include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophilic leukemia, blast cell leukemia, bovine leukemia, chronic myelocytic leukemia, leukemia cutis, embryonal leukemia, eosinophilic leukemia, Gross' leukemia, Rieder cell leukemia, Schilling's leukemia, stem cell leukemia, subleukemic leukemia, undifferentiated cell leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, plasmacytic leukemia, and promyelocytic leukemia.

The term “carcinoma” refers to a malignant new growth made up of epithelial cells tending to infiltrate the surrounding tissues, and/or resist physiological and non-physiological cell death signals and give rise to metastases. Exemplary carcinomas include, for example, /pindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrmcous carcinoma, carcinoma villosum, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, naspharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, and carcinoma scroti,

The term “sarcoma” generally refers to a tumor which is made up of a substance like the embryonic connective tissue and is generally composed of closely packed cells embedded in a fibrillar, heterogeneous, or homogeneous substance. Sarcomas include, chondrosarcoma, fibrosarcoma, lymphosarcoma, melanosarcoma, myxosarcoma, osteosarcoma, endometrial sarcoma, stromal sarcoma, Ewing's sarcoma, fascial sarcoma, fibroblastic sarcoma, giant cell sarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilns' tumor sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectaltic sarcoma. Additional exemplary neoplasias include, for example, Hodgkin's Disease, Non-Hodgkin's Lymphoma, multiple myeloma, neuroblastoma, breast cancer, ovarian cancer, lung cancer, rhabdomyosarcoma, primary thrombocytosis, primary macroglobulinemia, small-cell lung tumors, primary brain tumors, stomach cancer, colon cancer, malignant pancreatic insulanoma, malignant carcinoid, premalignant skin lesions, testicular cancer, lymphomas, thyroid cancer, neuroblastoma, esophageal cancer, genitourinary tract cancer, malignant hypercalcemia, cervical cancer, endometrial cancer, and adrenal cortical cancer.

The term “melanoma” is taken to mean a tumor arising from the melanocytic system of the skin and other organs. Melanomas include, for example, Harding-Passey melanoma, juvenile melanoma, lentigo maligna melanoma, malignant melanoma, acral-lentiginous melanoma, amelanotic melanoma, benign juvenile melanoma, Cloudman's melanoma, S91 melanoma, nodular melanoma subungal melanoma, and superficial spreading melanoma. The term “polypeptide” is used interchangeably with “peptide”, “altered peptide ligand”, and “flourocarbonated peptides.” The term “pharmaceutically acceptable carrier” includes any and all solvents, dispersion media, coatings, antibacterial and antifungal agents, isotonic and absorption delaying agents, and the like. The use of such media and agents for pharmaceutically active substances is well known in the art. Except insofar as any conventional media or agent is incompatible with the active compound, use thereof in the therapeutic compositions is contemplated. Supplementary active compounds can also be incorporated into the compositions.

The term “T cell” is also referred to as T lymphocyte, and means a cell derived from thymus among lymphocytes involved in an immune response. The T cell includes any of a CD8-positive T cell (cytotoxic T cell: CTL), a CD4-positive T cell (helper T cell), a suppressor T cell, a regulatory T cell such as a controlling T cell, an effector cell, a naive T cell, a memory T cell, an αβT cell expressing TCR α and β chains, and αγδT cell expressing TCR γ and δ chains. The T cell includes a precursor cell of a T cell in which differentiation into a T cell is directed. Examples of “cell populations containing T cells” include, in addition to body fluids such as blood (peripheral blood, umbilical blood etc.) and bone marrow fluids, cell populations containing peripheral blood mononuclear cells (PBMC), hematopoietic cells, hematopoietic stem cells, umbilical blood mononuclear cells etc., which have been collected, isolated, purified or induced from the body fluids. Further, a variety of cell populations containing T cells and derived from hematopoietic cells can be used in the present disclosure. These cells may have been activated by cytokine such as IL-2 in vivo or ex vivo. As these cells, any of cells collected from a living body, or cells obtained via ex vivo culture, for example, a T cell population obtained by the method of the present disclosure as it is, or obtained by freeze preservation, can be used.

The term “antibody” is meant to include both intact molecules as well as fragments thereof that include the antigen-binding site. Whole antibody structure is often given as H₂L₂ and refers to the fact that antibodies commonly comprise 2 light (L) amino acid chains and 2 heavy (H) amino acid chains. Both chains have regions capable of interacting with a structurally complementary antigenic target. The regions interacting with the target are referred to as “variable” or “V” regions and are characterized by differences in amino acid sequence from antibodies of different antigenic specificity. The variable regions of either H or L chains contains the amino acid sequences capable of specifically binding to antigenic targets. Within these sequences are smaller sequences dubbed “hypervariable” because of their extreme variability between antibodies of differing specificity. Such hypervariable regions are also referred to as “complementarity determining regions” or “CDR” regions. These CDR regions account for the basic specificity of the antibody for a particular antigenic determinant structure. The CDRs represent non-contiguous stretches of amino acids within the variable regions but, regardless of species, the positional locations of these critical amino acid sequences within the variable heavy and light chain regions have been found to have similar locations within the amino acid sequences of the variable chains. The variable heavy and light chains of all antibodies each have 3 CDR regions, each non-contiguous with the others (termed L1, L2, L3, H1, H2, H3) for the respective light (L) and heavy (H) chains. The antibodies according to the present disclosure may also be wholly synthetic, wherein the polypeptide chains of the antibodies are synthesized and, possibly, optimized for binding to the polypeptides disclosed herein as being receptors. Such antibodies may be chimeric or humanized antibodies and may be fully tetrameric in structure, or may be dimeric and comprise only a single heavy and a single light chain.

The term “effective amount” or “therapeutically effective amount” means a dosage sufficient to treat, inhibit, or alleviate one or more symptoms of a disease state being treated or to otherwise provide a desired pharmacologic and/or physiologic effect, especially enhancing T cell response to a selected antigen. The precise dosage will vary according to a variety of factors such as subject-dependent variables (e.g., age, immune system health, etc.), the disease, and the treatment being administered.

The terms “individual”, “host”, “subject”, and “patient” are used interchangeably herein, and refer to a mammal, including, but not limited to, primates, for example, human beings, as well as rodents, such as mice and rats, and other laboratory animals.

The term “treatment regimen” refers to a treatment of a disease or a method for achieving a desired physiological change, such as increased or decreased response of the immune system to an antigen or immunogen, such as an increase or decrease in the number or activity of one or more cells, or cell types, that are involved in such response, wherein said treatment or method comprises administering to an animal, such as a mammal, especially a human being, a sufficient amount of two or more chemical agents or components of said regimen to effectively treat a disease or to produce said physiological change, wherein said chemical agents or components are administered together, such as part of the same composition, or administered separately and independently at the same time or at different times (i.e., administration of each agent or component is separated by a finite period of time from one or more of the agents or components) and where administration of said one or more agents or components achieves a result greater than that of any of said agents or components when administered alone or in isolation.

As used herein, “tumor-associated immune suppression” refers to suppression of or evasion from the immune system by a tumor or cancer cell. Tumor-associated immune suppression may result from tumors or cancer cells that express immunoregulatory molecules, such as expressing CTLA-4, PD-1, or other checkpoint molecules, that block the function of immune cells against the tumor or cancer cell. Tumor-associated immune suppression may result from the tumor or cancer cell suppressing inflammatory signals to the immune system in order to evade immune-cytotoxicity. Tumor-associated immune suppression may result from the tumor or cancer cell modulating the tumor microenvironment or immune cells in the tumor microenvironment, such as antigen presenting cells or macrophages.

The term “anergy” and “unresponsiveness” includes unresponsiveness to an immune cell to stimulation, for example, stimulation by an activation receptor or cytokine. The anergy may occur due to, for example, exposure to an immune suppressor or exposure to an antigen in a high dose. Such anergy is generally antigen-specific, and continues even after completion of exposure to a tolerized antigen. For example, the anergy in a T cell and/or NK cell is characterized by failure of production of cytokine, for example, interleukin (IL)-2. The T cell anergy and/or NK cell anergy occurs in part when a first signal (signal via TCR or CD-3) is received in the absence of a second signal (costimulatory signal) upon exposure of a T cell and/or NK cell to an antigen.

The term “enhanced function of a T cell”, “enhanced cytotoxicity” and “augmented activity” means that the effector function of the T cell and/or NK cell is improved. The enhanced function of the T cell and/or NK cell, which does not limit the present disclosure, includes an improvement in the proliferation rate of the T cell and/or NK cell, an increase in the production amount of cytokine, or an improvement in cytotoxity. Further, the enhanced function of the T cell and/or NK cell includes cancellation and suppression of tolerance of the T cell and/or NK cell in the suppressed state such as the anergy (unresponsive) state, or the rest state, that is, transfer of the T cell and/or NK cell from the suppressed state into the state where the T cell and/or NK cell responds to stimulation from the outside. In some embodiments of the disclosure, immunogenic fibroblasts are utilized to induce immune responses, which result in breaking of anergy.

The term “expression” means generation of mRNA by transcription from nucleic acids such as genes, polynucleotides, and oligonucleotides, or generation of a protein or a polypeptide by transcription from mRNA. Expression may be detected by means including RT-PCR, Northern Blot, or in situ hybridization. “Suppression of expression” refers to a decrease of a transcription product or a translation product in a significant amount as compared with the case of no suppression. The suppression of expression herein shows, for example, a decrease of a transcription product or a translation product in an amount of 30% or more, preferably 50% or more, more preferably 70% or more, and further preferably 90% or more.

I. Cells of the Disclosure

Aspects of the disclosure concern the prior sensitization of tumors by administration of fibroblasts, in particular embodiments possess anti-inflammatory activity and are capable of reducing tumor-associated immune suppression. The anti-inflammatory activity in specific aspects includes suppressing the production of one or more inflammatory molecules, such as TNF-alpha, IL-1, IL-6, or a combination thereof. In some aspects, the inflammatory molecules may be suppressed in a tumor microenvironment (including adjacent cells) to the fibroblasts, such as cells adjacent to the tumor or tumor cells, including in a tumor microenvironment. In some embodiments, the fibroblasts are administered intra-tumorally and/or peritumorally in order to sensitize the tumor to immunological interventions. In some embodiments, subsequent to fibroblast-mediated tumor sensitization, systemic immunization is performed with tumor cells and/or tumor antigens, which is then, optionally, followed by induction of immunogenic cell death, optionally, followed by augmentation of tumor specific immune responses.

Fibroblasts useful for de-repressing of tumor immunity can be derived from various tissues, selected for specific properties associated with anti-inflammatory and/or immune stimulatory activity. Tissues useful for the practice of the disclosure are generally tissues associated with regenerative activity. The tissues include placenta, endometrial cells, Wharton's jelly, bone marrow, and adipose tissue, as examples. In some embodiments, the cells are selected for expression of the markers CD117 and/or CD105 and optionally possessing rhodamine 123 efflux activity. In some embodiments of the disclosure, fibroblasts are selected for based on the expression of markers including Oct-4, CD-34, KLF-4, Nanog, Sox-2, Rex-1, GDF-3, Stella, or a combination thereof. In some embodiments, the fibroblasts may or may not possess enhanced expression of GDF-11. Selection of fibroblasts for expression of the markers may be performed by initial expression of one or more proteins found on the membrane of the cells, which result in possessing other markers mentioned. In some embodiments of the invention, fibroblasts are selected for a marker, for example, CD-34. Selection may be performed by various means known in the art such as magnetic activated cell sorting (MACS), fluorescent activated cell sorting (FACS), immunopanning, or other means of selective adhesion. In some embodiments selection of cells possessing one marker results in selection of cells that express other markers. For example, selection of CD-34 expressing fibroblasts results in also selecting for fibroblasts that express higher rhodamine-123 efflux activity. The fibroblasts used for administration intratumorally and/or peritumorally are selected for by expression of anti-inflammatory properties, and these may include the ability to suppress (for example, with a bystander effect). TNF-alpha production from adjacent cells and/or the ability to suppress production of interleukins, such as IL 1 and/or IL-6.

In some embodiments, the fibroblasts are transfected with one or more immune stimulatory genes such as IL-1, IL-2, IL-7, IL-12, IL-15, IL-18, IL-21, IL-27, and IL-33. Alternatively, fibroblasts may be transfected with genes that increase accumulation of antigen presenting cells. Examples of such genes include G-CSF, GM-CSF, FLT-3 ligand, M-CSF, and a combination thereof. In other embodiments, fibroblasts are transfected with genes having inducible expression, such as any genes encompassed herein, for example using promoters such as the RheoSwitch® developed by Intrexon Corporation.

In some embodiments, the fibroblast are cultured in a manner to increase the activities or capabilities useful for the methods disclosed herein. The fibroblasts may be cultured to promote the ability of said fibroblasts to reduce inflammatory mediator production. In some embodiments, the fibroblast, including fibroblasts able to reduce inflammatory mediator production, are cultured in the presence of tissue culture additives, such as interleukin-10, indomethacin, valproic acid, low dose naltrexone, IL-27, or a combination thereof, for example. Inflammatory mediators may be selected from the group consisting of PGE-2, TNF-alpha, TNF-beta, interferon gamma, interleukin-33, interleukin-17, HMGB1, and a combination thereof.

In some embodiments, fibroblasts are dedifferentiated by any method known in the art. Fibroblasts may be subsequently re-differentiated after being dedifferentiated, which may induce the expression of one or more tumor antigens.

In some embodiments, fibroblasts express one or more tumor antigens including, for example, Fos-related antigen 1, LCK, FAP, VEGFR2, NA17, PDGFR-beta, PAP, MAD-CT-2, Tie-2, PSA, protamine 2, legumain, endosialin, prostate stem cell antigen, carbonic anhydrase IX, STn, Page4, proteinase 3, GM3 ganglioside, tyrosinase, MART1, gp100, SART3, RGS5, SSX2, Globol1, Tn, CEA, hCG, PRAME, XAGE-1, AKAP-4, TRP-2, B7H3, sperm fibrous sheath protein, CYP1B1, HMWMAA, sLe(a), MAGE A1, GD2, PSMA, mesothelin, fucosyl GM1, GD3, sperm protein 17, NY-ESO-1, PAX5, AFP, polysialic acid, EpCAM, MAGE-A3, mutant p53, ras, mutant ras, NY-BR1, PAX3, HER2/neu, OY-TES1, HPV E6 E7, PLAC1, hTERT, BORIS, ML-IAP, idiotype of b cell lymphoma or multiple myeloma, EphA2, EGFRvIII, cyclin B1, RhoC, androgen receptor, surviving, MYCN, wildtype p53, LMP2, ETV6-AML, MUC1, BCR-ABL, ALK, WT1, ERG (TMPRSS2 ETS fusion gene), sarcoma translocation breakpoint, STEAP, OFA/iLRP, Chondroitin sulfate proteoglycan 4 (CSPG4), Epithelial tumor antigen, alphafetoprotein, CD19, CA-125, or a combination thereof.

In some embodiments, prior to use the fibroblast cells may be cultured for at least between about 10 days and about 40 days, for at least between about 15 days and about 35 days, for at least between about 15 days and 21 days, such as for at least about 15, 16, 17, 18, 19 or 21 days. In some embodiments, the fibroblasts of the disclosure may be cultured for no longer than 60 days, or no longer than 50 days, or no longer than 45 days. The tissue explants and fibroblasts may be cultured in the presence of a liquid culture medium. Typically, the medium will comprise a basal medium formulation as known in the art. Many basal media formulations can be used to culture fibroblasts herein, including but not limited to Eagle's Minimum Essential Medium (MEM), Dulbecco's Modified Eagle's Medium (DMEM), alpha modified Minimum Essential Medium (alpha-MEM), Basal Medium Essential (BME), Iscove's Modified Dulbecco's Medium (IMDM), BGJb medium, F-12 Nutrient Mixture (Ham), Liebovitz L-15, DMEM/F-12, Essential Modified Eagle's Medium (EMEM), RPMI-1640, and modifications and/or combinations thereof. Compositions of the above basal media are generally known in the art, and it is within the skill of one in the art to modify or modulate concentrations of media and/or media supplements as necessary for the fibroblasts cultured. In some embodiments, a culture medium formulation may be explants medium (CEM) which is comprised of IMDM supplemented with 10% fetal bovine serum (FBS, Lonza), 100 U/ml penicillin G, 100 μg/ml streptomycin and 2 mmol/L L-glutamine (Sigma-Aldrich). Other embodiments may employ further basal media formulations, such as chosen from the ones above.

For use in the fibroblast culture, media can be supplied with one or more further components. For example, additional supplements can be used to supply the cells with the necessary trace elements and substances for optimal growth and expansion. Such supplements include insulin, transferrin, selenium salts, and combinations thereof. These components can be included in a salt solution such as, but not limited to, Hanks' Balanced Salt Solution (HBSS), Earle's Salt Solution. Further antioxidant supplements may be added, e.g., beta-mercaptoethanol. While many media already contain amino acids, some amino acids may be supplemented later, e.g., L-glutamine, which is known to be less stable when in solution. A medium may be further supplied with antibiotic and/or antimycotic compounds, such as, typically, mixtures of penicillin and streptomycin, and/or other compounds, exemplified but not limited to, amphotericin, ampicillin, gentamicin, bleomycin, hygromycin, kanamycin, mitomycin, mycophenolic acid, nalidixic acid, neomycin, nystatin, paromomycin, polymyxin, puromycin, rifampicin, spectinomycin, tetracycline, tylosin, and zeocin. Also contemplated is supplementation of cell culture medium with mammalian plasma or sera. Plasma or sera often contain cellular factors and components that are necessary for viability and expansion. The use of suitable serum replacements is also contemplated (e.g., FBS). In some embodiments, culturing tissue explants and fibroblast cells for time durations as defined herein, and preferably using media compositions as described herein results in the emergence and proliferation of a progenitor or stem cell of the disclosure. In some embodiments, fibroblast cells of the present disclosure are identified and characterized by their expression of one or more specific marker proteins, such as cell-surface markers. Detection and isolation of these cells can be achieved, e.g., through flow cytometry, ELISA, and/or magnetic beads. Reverse-transcription polymerase chain reaction (RT-PCR) can also be used to monitor changes in gene expression in response to differentiation. Methods for characterizing fibroblasts the present disclosure are provided herein. In certain embodiments, the marker proteins used to identify and characterize the fibroblasts are selected from the group consisting of c-Kit, Nanog, Sox2, Hey1, SMA, Vimentin, Cyclin D2, Snail, E-cadherin, Nkx2.5, GATA4, CD105, CD90, CD29, CD73, Wt1, CD34, CD45, and a combination thereof.

In some embodiments, the fibroblasts are cultured in a manner to increase the activities or capabilities useful for the methods disclosed herein. The fibroblasts may be cultured to promote the ability of the fibroblasts to reduce inflammatory mediator production. In some embodiments, the fibroblasts, including fibroblasts able to reduce inflammatory mediator production, are cultured in the presence of one or more tissue culture additives, such as interleukin-10, indomethacin, valproic acid, low dose naltrexone, interleukin-27, or a combination thereof, for example.

In some embodiments, the disclosure encompasses the further use of T cell modulator(s) (TCM) to enhance tumor inhibiting effects of fibroblasts. TCM is a pharmaceutical grade transfer factor, which activates T cells by reducing costimulatory requirements, thus potentially increasing infiltration of tumors by T cells. The concept of an immunologically acting “Transfer Factor” was originally identified by Henry Lawrence in a 1956 publication [276], in which he reported simultaneous transfer of delayed hypersensitivity to diphtheria toxin and to tuberculin in eight consecutive healthy volunteers who received extracts from washed leucocytes taken from the peripheral blood of tuberculin-positive, Schick-negative donors who were highly sensitive to purified diphtheria toxin and toxoid. The leucocyte extracts used for transfer contained no detectable antitoxin. The recipient subjects were Schick-positive (<0.001 unit antitoxin per ml. serum) and tuberculin-negative at the time of transfer. All the recipients remained Schick-positive for at least 2 weeks following transfer and in every case their serum contained less than 0.001 units antitoxin at the time when they exhibited maximal skin reactivity to toxoid. The “transfer factor” that was utilized was prepared by washing packed leukocytes isolated using the bovine fibrinogen method, and washing the leukocytes twice in recipient plasma. The washed leukocytes were subsequently lysed by 7-10 freeze-thaw cycles in the presence of DNAse with Mg++. Administration of the extract was performed intradermally and subcutaneously over the deltoid area.

Given that in those early days little was known regarding T cell specificity and MHC antigen presentation, the possibility that immunological information was transmitted by these low molecular weight transfer factors was taken seriously. Transfer factors of various sizes and charges were isolated, with some concept that different antigens elicited different types of transfer factors [277, 278]. Numerous theories were proposed to the molecular nature of transfer factor. Some evidence was that it constituted chains of antibodies that were preformed but subsequently cleaved [279]. Functionally, one of the main thoughts was that transfer factor has multiple sites of action, including effects on the thymus, on lymphocyte-monocyte and/or lymphocyte-lymphocyte interactions, as well as direct effects on cells in inflammatory sites. It is also suggested that the “specificity” of transfer factor is determined by the immunologic status of the recipient rather than by informational molecules in the dialysates [280].

Burger et al [281], used exclusion chromatography to perform characterization of transfer factor. The found that specific transferring ability of transfer factor in vivo was found to reside in the major UV-absorbing peak (Fraction III). Fraction III transferred tuberculin, candida, or KLH-reactivity to previously negative recipients. Fraction III from nonreactive donors was ineffective. When the fractions were tested in vitro, we found that both the mitogenic activity of whole transfer factor and the suppressive activity to mitogen activation when present in transfer factor was found in Fraction I. Fraction III contained components responsible for augmentation of PHA and PWM responses. In addition, Fraction III contained the component responsible for antigen-dependent augmentation of lymphocyte transformation. Fraction IV was suppressive to antigen-induced lymphocyte transformation.

In 1992 Kirkpatrick characterized the specific transfer factor at molecular level [280]. The transfer factor is constituted by a group of numerous molecules, of low molecular weight, from 1.0 to 6.0 kDa. The 5 kDa fraction corresponds to the transfer factor specific to antigens. There are a number of publications about the clinical indications of the transfer factor for diverse diseases, in particular those where the cellular immune response is compromised or in those where there is a deficient regulation of the immune response. It has been demonstrated that the transfer factor increases the expression of IFN-gamma and RANTES, while decreases the expression of osteopontine. Using animal models it has been reported that transfer factor possesses activity against M. tuberculosis, and with a model of glioma with good therapeutic results. In the clinical setting studies have reported effects against herpes zoster, herpes simplex type I, herpetic keratitis, atopic dermatitis, osteosarcoma, tuberculosis, asthma, post-herpetic neuritis, anergic coccidioidomycosis, leishmaniasis, toxoplasmosis, mucocutaneous candidiasis, pediatric infections produced by diverse pathogen germs, sinusitis, pharyngitis, and otits media. All of these diseases were studied through protocols which main goals were to study the therapeutic effects of the transfer factor, and to establish in a systematic way diverse dosage schema and time for treatment to guide the prescription of the transfer factor [282].

In some embodiments, dendritic cells (DC), as disclosed herein, are used to stimulate T cell and NK cell tumoricidal activity, for example by pulsing with autologous tumor lysate. Specifically, generated DC may be further purified from culture through use of flow cytometry sorting or magnetic activated cell sorting (MACS), or may be utilized as a semi-pure population. DC pulsed with tumor lysate may be administered into an individual, including an individual disclosed herein, which may stimulate NK and T cell activity in vivo, or in particular embodiments may be incubated in vitro with a population of cells containing T cells and/or NK cells. In some embodiments, DC are exposed to agents capable of stimulating maturation in vitro and rendering them resistant to tumor derived inhibitory compounds such as arginase byproducts. Specific means of stimulating in vitro maturation include culturing DC or DC containing populations with at least one toll like receptor agonist. Another means of achieving DC maturation involves exposure of DC to TNF-alpha at a concentration of approximately 20 ng/mL. In order to activate T cells and/or NK cells in vitro, cells are cultured in media containing approximately 1000 IU/ml of interferon gamma. Incubation with interferon gamma may be performed for the period of 2 hours to the period of 7 days. Preferably, incubation is performed for approximately 24 hours, after which T cells and/or NK cells are stimulated via the CD3 and CD28 receptors. One means of accomplishing this is by addition of antibodies capable of activating these receptors. In some embodiments, approximately, 2 ug/ml of anti-CD3 antibody is added, together with approximately 1 μg/ml anti-CD28. In order to promote survival of T cells and NK cells, as well as to stimulate proliferation, a T cell/NK mitogen may be used. In some embodiments, the cytokine IL-2 is utilized. Specific concentrations of IL-2 useful for the practice of the disclosure are approximately 500 u/mL IL-2. Media containing IL-2 and antibodies may be changed about every 48 hours for approximately 8-14 days. In one particular embodiment DC are included to the T cells and/or NK cells in order to endow cytotoxic activity towards tumor cells. In a particular embodiment, inhibitors of caspases are added in the culture so as to reduce rate of apoptosis of T cells and/or NK cells. In particular embodiments, generated cells can be administered to a subject intradermally, intramuscularly, subcutaneously, intraperitoneally, intraarterially, intravenously (including a method performed by an indwelling catheter), intratumorally, or into an afferent lymph vessel. The immune response of the individual treated with cytotoxic cells is assessed utilizing a variety of antigens found in tumor cells. When cytotoxic antibodies or antibodies associated with complement fixation are recognized to be upregulated in an individual, subsequent immunizations may be performed utilizing peptides to induce a focusing of the immune response.

In some embodiments, DC are generated from leukocytes of patients by leukopheresis. Numerous means of leukopheresis are known in the art. In one example, a Frenius Device (Fresenius Com.Tec) is utilized with the use of the MNC program, at approximately 1500 rpm, and with a P1Y kit. The plasma pump flow rates are adjusted to approximately 50 mL/min. Various anticoagulants may be used, for example ACD-A. The Inlet/ACD Ratio may be ranged from approximately 10:1 to 16:1. In some embodiments, approximately 150 mL of blood is processed. The leukopheresis product is subsequently used for initiation of dendritic cell culture.

In order to generate peripheral blood mononuclear cells from leukopheresis product, mononuclear cells are isolated by the Ficoll-Hypaque density gradient centrifugation. Monocytes are then enriched by the Percoll hyperosmotic density gradient centrifugation followed by two hours of adherence to the plate culture. Cells are then centrifuged at 500 g to separate the different cell populations. Adherent monocytes are cultured for 7 days in 6-well plates at 2×10⁶ cells/mL RMPI medium with 1% penicillin/streptomycin, 2 mM L-glutamine, 10% of autologous, 50 ng/mL GM-CSF and 30 ng/mL IL-4. On approximately day 6, immature dendritic cells may be pulsed with tumor antigen. Pulsing may be performed by incubation of lysates with dendritic cells, or may be generated by fusion of immature dendritic cells with tumor cells. Means of generating hybridomas or cellular fusion products are known in the art and include electrical pulse mediated fusion, or stimulation of cellular fusion by treatment with polyethelyne glycol. On day 7, the immature DCs are then induced to differentiate into mature DCs by culturing for 48 hours with 30 ng/mL interferon gamma (IFN-γ). During the course of generating DC for clinical purposes, microbiologic monitoring tests are performed at the beginning of the culture, on the fifth day and at the time of cell freezing for further use or prior to release of the dendritic cells.

In some embodiments, culture of the immune effectors cells, such as T cells, NK cells, and/or gamma delta T cells, is performed after extracting immune cells from a patient that has been immunized with a polyvalent antigenic preparation. Specifically separating the cell population and cell sub-population containing a T cell can be performed, for example, by fractionation of a mononuclear cell fraction by density gradient centrifugation, or a separation means using the surface marker of the T cell as an index. Subsequently, isolation based on surface markers may be performed. Examples of the surface marker include CD3, CD8 and/or CD4, and separation methods depending on these surface markers are known in the art. For example, the step can be performed by mixing a carrier such as beads or a culturing container on which an anti-CD8 antibody has been immobilized, with a cell population containing a T cell, and recovering a CD8-positive T cell bound to the carrier. As the beads on which an anti-CD8 antibody has been immobilized, for example, CD8 MicroBeads, Dynabeads M450 CD8, and Eligix anti-CD8 mAb coated nickel particles can be suitably used. This is also the same as in implementation using CD4 as an index and, for example, CD4 MicroBeads, Dynabeads M-450 CD4 can also be used.

In some embodiments of the disclosure, T regulatory cells are depleted before initiation of the culture. Depletion of T regulatory cells may be performed by negative selection by removing cells that express makers such as neuropilin, CD25, CD4, CTLA4, and membrane bound transforming growth factor (TGF)-beta. Experimentation by one of skill in the art may be performed with different culture conditions in order to generate effector lymphocytes, or cytotoxic cells, that possess both maximal activity in terms of tumor killing, as well as migration to the site of the tumor. For example, the step of culturing the cell population and cell sub-population containing a T cell can be performed by selecting suitable known culturing conditions depending on the cell population. In addition, in the step of stimulating the cell population, known proteins and chemical ingredients, etc., may be added to the medium to perform culturing. For example, cytokines, chemokines or other ingredients may be added to the medium. Herein, the cytokine is not particularly limited as far as how it can act on the T cell, and examples thereof include IL-2, IFN-gamma, TGF-beta, IL-15, IL-7, IFN-alpha, IL-12, CD40L, and IL-27. From the viewpoint of enhancing cellular immunity, particularly suitably, IL-2, IFN-gamma, or IL-12 may be used and, from the viewpoint of improvement in survival of a transferred T cell in vivo, IL-7, IL-15 or IL-21 may be suitably used. In addition, the chemokine is not particularly limited as far as how it acts on the T cell and exhibits migration activity, and examples thereof include RANTES, CCL21, MIP1alpha, MIP1beta, CCL19, CXCL12, IP-10 and MIG.

The stimulation of the cell population can be performed by the presence of a ligand for a molecule present on the surface of the T cell, for example, CD3, CD28, or CD44 and/or an antibody to the molecule. Further, the cell population can be stimulated by contacting with other lymphocytes such as antigen presenting cells (dendritic cell) presenting a target peptide such as a peptide derived from a cancer antigen on the surface of a cell. In addition to assessing cytotoxicity and migration as end points, it is within the scope of the current disclosure to optimize the cellular product based on other means of assessing T cell activity, for example, the functional enhancement of the T cell in the method of the present disclosure may be assessed at a plurality of time points before and after each step, including using a cytokine assay, an antigen-specific cell assay (tetramer assay), a proliferation assay, a cytolytic cell assay, an in vivo delayed hypersensitivity test using a recombinant tumor-associated antigen or an immunogenic fragment or an antigen-derived peptide, or a combination thereof.

Examples of an additional method for measuring an increase in an immune response include a delayed hypersensitivity test, flow cytometry using a peptide major histocompatibility gene complex tetramer, a lymphocyte proliferation assay, an enzyme-linked immunosorbent assay, an enzyme-linked immunospot assay, cytokine flow cytometry, a direct cytotoxity assay, measurement of cytokine mRNA by a quantitative reverse transcriptase polymerase chain reaction, or an assay which is currently used for measuring a T cell response such as a limiting dilution method. In vivo assessment of the efficacy of the generated cells used in the disclosure may be assessed in a living body before first administration of the T cell with enhanced function of the present disclosure, or at various time points after initiation of treatment, using an antigen-specific cell assay, a proliferation assay, a cytolytic cell assay, an in vivo delayed hypersensitivity test using a recombinant tumor-associated antigen or an immunogenic fragment or an antigen-derived peptide, or a combination thereof. Further, an immune response can be assessed by a weight, diameter or malignant degree of a tumor possessed by a living body, or the survival rate or survival term of a subject or group of subjects. The cells can be expanded in the presence of specific antigens associated with tumors and subsequently injected into the patient in need of treatment. Expansion with specific antigens includes co-culture with proteins including ROBO, VEGF-R2, FGF-R, CD105, TEM-1, survivin, or a combination thereof. Other tumor specific or semi-specific antigens are known in the art that may be used.

Subsequent to augmentation of lymphocyte numbers specific for killing of the tumor, modification of the tumor microenvironment may be performed. In some embodiments, macrophage modulators are used. Macrophages are key components of the innate immune system which play a principal role in the regulation of inflammation as well as physiological processes such as tissue remodeling [42, 43]. The diverse role of macrophages can be seen in conditions ranging from wound healing [44-47], to myocardial infarction [48-54], to renal failure [55-58] and liver failure [59].

Differentiated macrophages and their precursors are versatile cells that can adapt to micro-environmental signals by altering their phenotype and function [60]. Although they have been studied for many years, it has only recently been shown that these cells comprise distinct sub-populations, known as classical M1 and alternative M2 [61]. Mirroring the nomenclature of Th1 cells, M1 macrophages are described as the pro-inflammatory sub-type of macrophages induced by IFN-.gamma. and LPS. They produce effector molecules (e.g., reactive oxygen species) and pro-inflammatory cytokines (e.g., IL-12, TNF-.alpha. and IL-6) and they trigger Th1 polarized responses [62].

Macrophages can play a tumor inhibitory, as well as a tumor stimulatory role. Initial studies supported the role of macrophages in mediating antibody dependent cellular cytotoxicity in tumors [63-70], and thus being associated with potentiation of antitumor immune responses. Macrophages also possess the ability to directly recognize tumors by virtue of tumor expressed “eat-me” signals, which include the stress associated protein calreticulin [71, 72], which binds to the low-density lipoprotein receptor-related protein (LRP) on macrophages to induce phagocytosis [73]. Tumors protect themselves by expression of CD47, which binds to macrophage SIRP-1 and transduces an inhibitory signal [74]. Blockade of CD47 using antibodies results in remission of cancers mediated by macrophage activation [75-79]. Thus on the one hand, macrophages play an important role in induction of antitumor immunity. This can also be exemplified by some studies, involving administration of GM-CSF in order to augment macrophage numbers and activity in cancer patients [80-83].

Unfortunately, there is also evidence that macrophages support tumor growth. Studies in the osteopetrotic mice strain, which lacks mature macrophages, demonstrate that tumors actually grow slower in animals deficient in macrophages [84]. Several other animal models have elegantly demonstrated that macrophages contribute to tumor growth, in part through stimulating on the angiogenic switch [85-87]. Numerous tumor biopsy studies have shown that there is a negative correlation between macrophage infiltration into tumors and patient survival [88-92].

The duality of macrophages in growth of tumors may be seen in studies of “inverse hormesis” in which low concentrations of antibodies targeting the tumor specific marker sialic acid N-glycolyl-neuraminic acid (Neu5Gc) actually leads to enhanced tumor growth in a macrophage dependent manner [93].

The importance of macrophages in clinical implementation of cancer therapeutics can be seen from results of a double blind clinical trials in metastatic colorectal cancer patients where cetuximab (anti-epidermal growth factor receptor (EGFR) monoclonal antibody (mAb)) was added to a protocol comprising of bevacizumab and chemotherapy. The addition of cetuximab actually resulted in decreased survival. In a study examining whether monocyte conversion to M2 angiogenic macrophages was responsible, investigators observed that CD163-positive M2 macrophages were found in high concentrations intratumorally in patients with colorectal carcinomas. These M2 cells expressed abundant levels of Fc-gamma receptors (FcγR) and PD-L1. Additionally, consistent with the M2 phenotype the cells generated large amounts of the immunosuppressive molecule IL-10 and the angiogenic mediator VEGF. When M2 cells were cultured with EGFR-positive tumor cells loaded with low concentrations of cetuximab, further augmentation of IL-10 and VEGF production was observed. These data suggest that under certain contexts, tumors manipulate macrophages to take on the M2 phenotype, and this subsequently leads to enhanced tumor progressing factors when tumor cells are bound by antibodies [94].

Manipulation of macrophages to inhibit M2 and shift to M1 phenotype may be performed using a variety of means. One theme that may be unifying is the ability of toll like receptor (TLR) agonists to influence this. In addition to cytokine differences, macrophages capable of killing tumor cells are usually known to express low levels of the inhibitory Fc gamma receptor IIb, whereas tumor promoting macrophages have high levels of this receptor [95]. Furthermore, tumor associated cytokines such as IL-4 and IL-10 are known to induce upregulation of the Fc gamma receptor IIB [96-99].

In one study, the effect of the TLR7/8 agonist R-848 was assessed on monocytes derived from human peripheral blood. It was found that 12 hour exposure of R-848 increased FcγR-mediated cytokine production and antibody-dependent cellular cytotoxicity by monocytes. Furthermore, upregulation of the ADCC associated receptors FcγRI, FcγRIIa, and the common gamma-subunit was observed. However treatment with R-848 led to profound downregulation of the inhibitory FcγRIIb molecule [100]. These data support ability to modify therapeutic activity of macrophages by manipulation of TLR signaling pathways. Other TLRs have been found to suppress inhibitory receptors on macrophages. For example, in another study it was observed that exposing monocytes to TLR4 agonists leads to suppression of the FcγRIIb macrophage inhibitory protein by MARCH3 mediated ubiquitination [101].

In one embodiment administration of ImmunoMax is performed systemically, and/or locally, which is an injectable polysaccharide purified from potato sprouts and approved as pharmaceutical in the Russian Federation (registration P No. 001919/02-2002) and 5 other countries of Commonwealth of Independent States (formerly the USSR) and has been evaluated in a wide range of medical situations. In accordance with the formal “Instruction of Medical Use”, one medical indication for Immunomax® is the stimulation of immune defense during the treatment of different infectious diseases (http://www.gepon.ru/immak_intro.htm). Studies have shown that Immunomax® induces immune mediated killing of cancer cells in a TLR4 dependent manner [102]. In some embodiments of the disclosure, ImmunoMax is utilized to induce an M2 to M1 shift, thus reducing macrophage derived immune suppressants and augmenting production of immune stimulatory cytokines such as IL-12 and TNF-alpha [102]. In some embodiments of the disclosure, other agents may be used to modulate M2 to M1 transition of tumor associated macrophages including RRx-001 [103], the bee venom derived peptide melittin [104], CpG DNA [105, 106], metformin [107], the Chinese medicine derivative puerarin [108], the rhubarb derivative emodin [109], dietary supplement chlorogenic acid [110], propranolol [111], poly ICLC [112], BCG [113], Agaricus blazei Murill mushroom extract [114], endotoxin [115], olive skin derivative maslinic acid [116], intravenous immunoglobulin [117], phosphotidylserine targeting antibodies [118], dimethyl sulfoxide [119], surfactant protein A [120], zoledronic acid [121], and bacteriophages [122]. In some embodiments, fibroblasts are administered to reprogram macrophages from M2 to M1, in such cases fibroblasts may be immunogenic fibroblasts, and/or fibroblasts transfected with immune cytokines that promote M1 and suppress M2. Such immune cytokines include IL-2, IL-12, interferon gamma, IL-18, IL-27 and IL-33.

II. Immunogenic Compositions

Particular aspects of the disclosure concern immunogenic compositions of any kind, including vaccines, that comprise peptides, antigens, lipids, carbohydrates, lipoproteins, proteoglycans, nucleic acid product, or the like that are expressed or appear on a cancer afflicting an individual, including an individual of the present disclosure. The immunogenic composition may induce an immune response of any kind, including the expansion of immune cells with tumor-targeting ability in an individual, including an individual of the present disclosure. The immunogenic composition may be matched to the HLA haplotype of an individual, including an individual of the present disclosure.

In some embodiments, the immunogenic composition is derived from a tumor or cancer cells from an individual, including an individual of the present disclosure, or from a tumor or cancer cells that are histologically similar, such as a tumor or cancer cells that are of the same sub-type, to an individual afflicted with or at risk for cancer. The immunogenic composition may comprise a molecule, or be derived from a molecule, extracted from a tumor or cancer cells, or histologically similar tumor or cancer cell. The molecule may be an mRNA, protein (including any peptide thereof), exosome, lipid, carbohydrate, lipoprotein, proteoglycan, nucleic acid product, or the like. The molecule may be extracted from the tumor or cancer cell by any method known in the art, including lysis, mRNA extraction, exosome extraction, or a combination thereof.

In some embodiments, the immunogenic composition comprises a polyvalent tumor vaccine, such as CanVaxin [28, 29], or other polyvalent vaccine mixtures. Numerous tumor antigens can be utilized to amplify the immune response selectively, and these can be chosen from known groups of tumor antigens or tumor associated proteins, such as ERG, WT1, ALS, BCR-ABL, Ras-mutant, MUC1, ETV6-AML, LMP2, p53 non-mutant, MYC-N, surviving, androgen receptor, RhoC, cyclin B1, EGFRvIII, EphA2, B cell or T cell idiotype, ML-IAP, BORIS, hTERT, PLAC1, HPV E6, HPV E7, OY-TES1, Her2/neu, PAX3, NY-BR-1, p53 mutant, MAGE A3, EpCAM, polysialic Acid, AFP, PAX5, NY-ESO1, sperm protein 17, GD3, Fucosyl GM1, mesothelin, PSMA, GD2, MAGE A1, sLe(x), HMWMAA, CYP1B1, sperm fibrous sheath protein, B7H3, TRP-2, AKAP-4, XAGE 1, CEA, Tn, GloboH, SSX2, RGS5, SART3, gp100, MelanA/MART1, Tyrosinase, GM3 ganglioside, Proteinase 3 (PRI), Page4, STn, Carbonic anhydrase IX, PSCA, Legumain, MAD-CT-1 (protamin2), PSA, Tie 2, MAD-CT2, PAP, PDGFR-beta, NA17, VEGFR2, FAP, LCK, Fos-related antigen, LCK, FAP, or a combination thereof.

In certain embodiments, the tumor antigen may be any peptide derived from a tumor associated protein (including a peptide comprising the entire protein) selected from the group consisting of Fos-related antigen 1, LCK, FAP, VEGFR2, NA17, PDGFR-beta, PAP, MAD-CT-2, Tie-2, PSA, protamine 2, legumain, endosialin, prostate stem cell antigen, carbonic anhydrase IX, STn, Page4, proteinase 3, GM3 ganglioside, tyrosinase, MART1, gp100, SART3, RGS5, SSX2, Globol1, Tn, CEA, hCG, PRAME, XAGE-1, AKAP-4, TRP-2, B7H3, sperm fibrous sheath protein, CYP1B1, HMWMAA, sLe(a), MAGE A1, GD2, PSMA, mesothelin, fucosyl GM1, GD3, sperm protein 17, NY-ESO-1, PAX5, AFP, polysialic acid, EpCAM, MAGE-A3, mutant p53, ras, mutant ras, NY-BR1, PAX3, HER2/neu, OY-TES1, HPV E6 E7, PLAC1, hTERT, BORIS, ML-IAP, idiotype of b cell lymphoma or multiple myeloma, EphA2, EGFRvIII, cyclin B1, RhoC, androgen receptor, surviving, MYCN, wildtype p53, LMP2, ETV6-AML, MUC1, BCR-ABL, ALK, WT1, ERG (TMPRSS2 ETS fusion gene), sarcoma translocation breakpoint, STEAP, OFA/iLRP, Chondroitin sulfate proteoglycan 4 (CSPG4), Epithelial tumor antigen, alphafetoprotein, CD19, CA-125, and a combination thereof.

In some embodiments, the immunogenic composition is administered to an individual, including an individual of the present disclosure, in combination with an adjuvant. The adjuvant may stimulate antigen presentation. The adjuvant may comprise a toll like receptor (TLR) including TLR-2 (which may be activated by Pam3cys4, heat killed Listeria monocytogenes, FSL-1, or a combination thereof), TLR-3 (which may be activated by Poly IC, double stranded RNA, or both), TLR-4 (which may be activated by lipopolysaccharide, HMGB-1 (or peptide derived thereof), or both), TLR-5 (which may be activated by flagellin), TLR-7 (which may be activated by imiquimod), TLR-8 (which may be activated by resmiquimod), TLR-9 (which may be activated by CpG DNA), or a combination thereof. The double stranded RNA may be of any origin, including mammalian and/or prokaryotic origins. In some embodiments, the double stranded RNA is from freeze-thawed leukocytes. The double stranded RNA may comprise freeze-thawed leukocyte extract that has been dialyzed for compositions less than 15 kDa. Any method known in the art for free-thawing leukocytes and dialyzing for compositions less than 15 kDa may be used. The peptide derived from HMGB-1 may be hp91. In some embodiments, TLR-4 is activated by a peptide comprising at least 80, 85, 90, 95, 96, 97, 98, 99, or 100 percent identity to the peptide with an amino acid sequence of

(SEQ ID NO: 1)
EFDVILKAAGANKVAVIKAVRGATGLGLKEAKDLVE
SAPAALKEGVSKDDAEALKKALEEAGAEVEVK.

Within the context of the disclosure, teachings are provided to amplify an antigen specific immune response following immunization with a polyvalent vaccine, in which the antigenic epitopes are used for immunization together with adjuvants such as toll like receptors (TLRs). These molecules are type 1 membrane receptors that are expressed on hematopoietic and non-hematopoietic cells. At least 11 members have been identified in the TLR family. These receptors are characterized by their capacity to recognize pathogen-associated molecular patterns (PAMP) expressed by pathogenic organisms. It has been found that triggering of TLR elicits profound inflammatory responses through enhanced cytokine production, chemokine receptor expression (CCR2, CCR5 and CCR7), and costimulatory molecule expression. As such, these receptors in the innate immune systems exert control over the polarity of the ensuing acquired immune response. Among the TLRs, TLR9 has been extensively investigated for its functions in immune responses. Stimulation of the TLR9 receptor directs antigen-presenting cells (APCs) towards priming potent, T_{H1}-dominated T-cell responses, by increasing the production of pro-inflammatory cytokines and the presentation of co-stimulatory molecules to T cells. CpG oligonucleotides, ligands for TLR9, were found to be a class of potent immunostimulatory factors. CpG therapy has been tested against a wide variety of tumor models in mice, and has consistently been shown to promote tumor inhibition or regression.

In some embodiments, the adjuvant that stimulates antigen presentation may increase the expression of at least one costimulatory molecule on antigen presenting cells, such as dendritic cells. The costimulatory molecule may comprise any molecule that stimulates antigen presentation, such as CD40, CD80, CD86, or a combination thereof, for example. The adjuvant increasing the expression of at least one costimulatory molecules may comprise an activator of NF-kappa-B such as an inhibitor of i-kappa-B, an activator a PAMP receptor, or other NF-kappa-B activators. The PAMP receptor may be any PAMP receptor including MDA5, RIG-1, NOD, or a combination thereof. The adjuvant increasing the expression of at least one costimulatory molecules may comprise an activator of the JAK-STAT pathway. The JAK-STAT activator may be any activator of the JAK-STAT pathway, including interferon gamma.

The combination of polyvalent vaccines with other cellular therapies as the initial poly-immunogenic composition is envisioned within the context of the disclosure. In some embodiments, cellular lysates of tumor cells, or tumor stem cells are loaded into dendritic cells. In some embodiments, fibroblasts are utilized as the basis for generating a hybridoma with autologous and/or allogeneic tumor cells, and the hybridoma may subsequently be utilized as a source of antigens for loading of dendritic cells. In some embodiments, the disclosure provides a means of generating a population of cells with tumoricidal ability that are polyvalently reactive, to which focus is added by subsequent peptide specific vaccination. The generation of cytotoxic lymphocytes may be performed, in one embodiment, by extracting approximately 50 ml of peripheral blood from a cancer patient and peripheral blood monoclear cells (PBMC) are isolated using the Ficoll Method. PBMCs are subsequently resuspended in 10 ml AIM-V media and allowed to adhere onto a plastic surface for approximately 2-4 hours. The adherent cells are then cultured at 37° C. in AIM-V media supplemented with approximately 1,000 U/mL granulocyte-monocyte colony-stimulating factor and approximately 500 U/mL IL-4 after non-adherent cells are removed by gentle washing in Hanks Buffered Saline Solution (HBSS). Half of the volume of the GM-CSF and IL-4 supplemented media is changed every other day. Immature DCs are harvested on approximately day 7.

In some embodiments of the disclosure, specific antigens are used for immunization following polyvalent immunization, and the specific antigens administered in the form of DNA vaccines. Specific antigens are generally tumor associated antigens such as telomerase, p53, ras, raf, GAGE, MAGE, BAGE, BORIS and NR2F6. Numerous publications have reported animal and clinical efficacy of DNA vaccines which are incorporated by reference [30-32]. In addition to direct DNA injection techniques, DNA vaccines can be administered by electroporation [33]. The nucleic acid compositions, including the DNA vaccine compositions, may further comprise a pharmaceutically acceptable excipient. Examples of suitable pharmaceutically acceptable excipients for nucleic acid compositions, including DNA vaccine compositions, are well known to those skilled in the art and include sugars, etc. Such excipients may be aqueous or non-aqueous solutions, suspensions, and emulsions. Examples of non-aqueous excipients include propylene glycol, polyethylene glycol, vegetable oils such as olive oil, and injectable organic esters such as ethyl oleate. Examples of aqueous excipient include water, alcoholic/aqueous solutions, emulsions or suspensions, including saline and buffered media. Suitable excipients also include agents that assist in cellular uptake of the polynucleotide molecule. Examples of such agents are (i) chemicals that modify cellular permeability, such as bupivacaine, (ii) liposomes or viral particles for encapsulation of the polynucleotide, or (iii) cationic lipids or silica, gold, or tungsten microparticles which associate themselves with the polynucleotides. Anionic and neutral liposomes are well-known in the art (see, e.g., Liposomes: A Practical Approach, RPC New Ed, IRL press (1990), for a detailed description of methods for making liposomes) and are useful for delivering a large range of products, including polynucleotides. Cationic lipids are also known in the art and are commonly used for gene delivery. Such lipids include Lipofectin™ also known as DOTMA (N-[I-(2,3-dioleyloxy) propyls N,N, N-trimethylammonium chloride), DOTAP (1,2-bis (oleyloxy)-3 (trimethylammonio) propane), DDAB (dimethyldioctadecyl-ammonium bromide), DOGS (dioctadecylamidologlycyl spermine) and cholesterol derivatives such as DCChol (3 beta-(N-(N′,N′-dimethyl aminomethane)-carbamoyl) cholesterol). A description of these cationic lipids can be found in EP 187,702, WO 90/11092, U.S. Pat. No. 5,283,185, WO 91/15501, WO 95/26356, and U.S. Pat. No. 5,527,928.

A particularly useful cationic lipid formulation that may be used with the nucleic vaccine provided by the disclosure is VAXFECTIN, which is a co-mixture of a cationic lipid (GAP-DMORIE) and a neutral phospholipid (DPyPE) which, when combined in an aqueous vehicle, self-assemble to form liposomes. Cationic lipids for gene delivery are preferably used in association with a neutral lipid such as DOPE (dioleyl phosphatidylethanolamine), as described in WO 90/11092 as an example. In addition, a DNA vaccine can also be formulated with a nonionic block copolymer such as CRL1005. Other immunization means include prime boost regiments [34].

III. Augmenting Antigen Presentation

Certain embodiments of the disclosure concern the administration of compositions that augment antigen presentation, are capable of augmenting antigen presentation, and/or activate antigen presentation locally. The composition may comprise a dendritic cell. In some embodiments, prior to induction of immunogenic cell death, antigen presenting cells are administered within the current disclosure, one of the most potent antigen presenting cells is the dendritic cell. Dendritic cells of the present disclosure may be from any source, including an autologous and/or allogenic source relative to an individual, including an individual of the present disclosure. The dendritic cell may be derived from another cell type, including a monocyte. The dendritic cell may be activated by a TLR agonist, a PAMP agonist, in vivo administration of GM-CSF, in vivo administration of FLT-3L, or a combination thereof.

Dendritic cells (DC) possess unique morphology similar to neuronal dendrites and were originally identified based on their ability to stimulate the adaptive immune system. Of importance to the field of tumor immunotherapy, dendritic cells appear to be the only cell in the body capable of activating naïve T cells [123]. The concept of dendritic cells instructing naïve T cells to differentiate into effector or memory cells is fundamental because it places the dendritic cell as the most powerful antigen presenting cell. This implies that for immunotherapeutic purposes, dendritic cells do not necessarily need to be administered at high numbers in patients. One way in which dendritic cells have been described is as sentinels of the immune system that are patrolling the body in an immature state [124, 125]. Once DC are activated, by a stimulatory signal such as a Damage Associated Molecular Patterns (DAMPs), the DC then migrate into the draining lymph nodes through the afferent lymphatics. During the trafficking process, DC degrade ingested proteins into peptides that bind to both MHC class I molecules and MHC class II molecules. This allows the DC to: a) perform cross presentation in that they ingest exogenous antigens but present peptides in the MHC I pathway; and b) activate both CD8 (via MHC I) and CD4 (via MHC II). Interestingly, lipid antigens are processed via different pathways and are loaded onto non-classical MHC molecules of the CD1 family [126].

The possibility of utilizing DC to stimulate immunity was made into reality in animal studies that took advantage of the ability of immature DC to potently phagocytose various antigens. If the antigens possessed DAMPs, or if DAMPs were present in the environment, the DC would mature and present the antigens, resulting in stimulation of potent T cell immunity. Accordingly, in the initial studies, immature DC were incubated with various antigens, subsequent to which a maturation signal (replicating natural DAMPs) was applied and the DC were injected into animals. Thus DC were utilized as a type of “cellular adjuvant”. Indeed, it was discovered that the classical adjuvants such as Fruend's Adjuvant actually contained a high concentration of DAMPs, which resulted in the stimulation of local DC at vaccination site in vivo.

One of the first clinical applications of DC was prostate cancer. In an early reported, thirty three androgen resistant metastatic prostate cancer patients were treated with DC that were pulsed with peptides from a prostate specific antigen termed PMSA. Nine partial responders were identified based on NCPC criterial, plus 50% reduction of PSA. Four of the partial responders were also responders in the phase I study, with an average response duration of 225 days. Their combined average total response period was over 370 days. Five other responders in the secondary immunizations at the Phase II were nonresponders in the phase I study. Their average partial response period was 196 days. These data support the safety of follow-up infusion of DC that have been pulsed with tumor antigen derived peptide [127].

The same group published a subsequent paper on an additional 33 patients that had not received prior DC immunization in the Phase I. All subjects received six infusions of DC pulsed with PSM-P1 and -P2 at six week intervals without any treatment associated adverse events. Six partial and two complete responders were identified in the phase II study based on NPCP criteria, plus 50% reduction of prostate-specific antigen (PSA), or resolution in previously measurable lesions on ProstaScint scan [128]. The same group analyzed immune response in patients who had clinical remission or relapsed. A strong correlation was found between delayed type hypersensitivity response to the PSM-P1 and PSM-P2 and clinical response [129].

Another subsequent study utilized DC generated using GM-CSF and IL-4 but pulsed with PAP, another prostate antigen. Specifically, the PAP was delivered to the DC by means of generation of a PAP-GM-CSF fusion protein. Two intravenous infusions of the generated cells were performed one month apart in 12 patients with androgen resistant prostate cancer. The infusions were followed by three s.c. monthly doses of the fusion protein without cells. Treatment was well tolerated and circulating prostate-specific antigen levels dropped in three patients. Immune response to the fusion protein was observed, as well as to PAP [130]. In addition to prostate cancer, in which FDA approval has been granted for the Provenge drug, numerous trials have been conducted in a wide variety of cancers. All the trials demonstrated safety, without serious adverse effects of DC administration, as well as some degree of therapeutic efficacy. Trials have been conducted in melanoma [131-182], soft tissue sarcoma [183], thyroid [184-186], glioma [187-208], multiple myeloma, [209-217], lymphoma [218-220], leukemia [221-228], as well as liver [229-234], lung [235-248], ovarian [249-252], and pancreatic cancer [253-255].

Within the context of the disclosure, T cell activation may be performed in vivo. In some embodiments, a transfer factor is utilized. T cells are immune effectors against tumors, possessing ability to directly kill via CD8 cytotoxic cells [256-258], or indirectly killing tumors by activation of macrophages through interferon gamma production [259-261]. Additionally, T cells have been shown to convert protumor M2 macrophages to M1 [262]. The importance of T cells in cancer is illustrated by positive correlation between tumor infiltrating lymphocytes and patient survival [263-267]. In addition, positive correlations between responses to various immunotherapies has been made with tumor infiltrating lymphocyte density [268, 269]. Increased T cell activity is associated with reduction in T regulatory (Treg) cells. Studies show that agents that cause suppression of Treg cells correlates with improved tumor control. Agents that inhibit Treg cells include arsenic trioxide [270], cyclophosphamide [271-273], triptolide [272], gemcitabine [274], and artemether [275].

IV. Compositions and Activity Capable of Inducing Cancer Cell Death

Particular embodiments of the present disclosure concern the administration of one or more compositions and/or activities or conditions that induce cancer cell death in an individual and/or are capable of inducing cancer cell death. Any composition and/or activity and/or condition for inducing cancer cell death known in the art may be used. Cancer cell death may be induced by an action. The action may comprise the administration of radiation therapy, including localized radiation therapy. The radiation therapy may be external beam radiation therapy and/or internal radiation therapy. In some embodiments, the action may comprise the administration of cryoablation therapy, including any method for administration of cryogenic compositions known in the art. The administration of cryoablation therapy may comprise administration of liquid nitrogen and/or argon gas. The administration of cryotherapy may comprise the administration of a cryogenic composition, such as a composition cooled to less than −30° C. including approximately −30° C., −35° C., −40° C., −45° C., −50° C., −55° C., −60° C., −65° C., −70° C., −75° C., −80° C. or below. The administration of cryoablation therapy may be intratumoral.

The activity or condition may comprise the exposure of the cancer to temperatures higher than normally present in the individual, which may comprise exposing the cancer cells to hyperthermia. The hyperthermia may be local, regional, and/or whole body hyperthermia. The cancer cells may be exposed to temperatures higher than 102° C., 103° C., 104° C., 105° C., 106° C., 107° C., 108° C., 109° C., 110° C., 111° C., 112° C., 113° C., or higher. The hyperthermia may increase the susceptibility of the cancer cells to other treatment and/or induce cancer cell death directly. Any method for exposing cancer cells to higher than normal temperatures may be used including, exposure to radio waves, microwaves, ultrasonic waves (including high intensity focused ultrasound or focused ultrasound), or other forms of energy. In some embodiments, the regional areas of the body, such as a region that is afflicted by cancer and/or a tumor, are exposed to hyperthermia. Regional exposure to hyperthermia may comprise regional and/or isolated perfusion using a heating device that may heat blood externally then return the blood to the circulation. In some embodiments, the entire body is exposed to hyperthermia.

The composition that induces cancer cell death may comprise a chemotherapy, targeted therapy, or other cancer therapy including, but not limited to, acivicin, aclarubicin, acodazole hydrochloride, acronine, adozelesin, aldesleukin, altretamine, ambomycin, ametantrone acetate, aminoglutethimide, amsacrine, anastrozole, anthramycin, asparaginase, asperlin, azacitidine, azetepa, azotomycin, batimastat, benzodepa, bicalutamide, bisantrene hydrochloride, bisnafide dimesylate, bizelesin, bleomycin sulfate, brequinar sodium, bropirimine, busulfan, cactinomycin, calusterone, caracemide, carbetimer, carboplatin, carmustine, carubicin hydrochloride, carzelesin, cedefingol, chlorambucil, cirolemycin, cisplatin, cladribine, crisnatol mesylate, cyclophosphamide, cytarabine, dacarbazine, dactinomycin, daunorubicin hydrochloride, decitabine, dexormaplatin, dezaguanine, dezaguanine mesylate, diaziquone, docetaxel, doxorubicin, doxorubicin hydrochloride, droloxifene, droloxifene citrate, dromostanolone propionate, duazomycin, edatrexate, eflornithine hydrochloride, elsamitrucin, enloplatin, enpromate, epipropidine, epirubicin hydrochloride, erbulozole, esorubicin hydrochloride, estramustine, estramustine phosphate sodium, etanidazole, etoposide, etoposide phosphate, etoprine, fadrozole hydrochloride, fazarabine, fenretinide, floxuridine, fludarabine phosphate, fluorouracil, fluorocitabine, fosquidone, fostriecin sodium, gemcitabine, gemcitabine hydrochloride, hydroxyurea, idarubicin hydrochloride, ifosfamide, ilmofosine, interleukin II (including recombinant interleukin II, or rIL2), interferon alfa-2a, interferon alfa-2b, interferon alfa-n1, interferon alfa-n3, interferon beta-I a, interferon gamma-I b, iproplatin, irinotecan hydrochloride, lanreotide acetate, letrozole, leuprolide acetate, liarozole hydrochloride, lometrexol sodium, lomustine, losoxantrone hydrochloride, masoprocol, maytansine, mechlorethamine hydrochloride, megestrol acetate, melengestrol acetate, melphalan, menogaril, mercaptopurine, methotrexate, methotrexate sodium, metoprine, meturedepa, mitindomide, mitocarcin, mitocromin, mitomalcin, mitomycin, mitosper, mitotane, mitoxantrone hydrochloride, mycophenolic acid, nocodazole, nogalamycin, ormaplatin, oxisuran, paclitaxel, pegaspargase, peliomycin, pentamustine, peplomycin sulfate, perfosfamide, pipobroman, piposulfan, piroxantrone hydrochloride, plicamycin, plomestane, porfimer sodium, porfiromycin, prednimustine, procarbazine hydrochloride, puromycin, puromycin hydrochloride, pyrazofurin, riboprine, rogletimide, safingol, safingol hydrochloride, semustine, simtrazene, sparfosate sodium, sparsomycin, spirogermanium hydrochloride, spiromustine, spiroplatin, streptonigrin, streptozocin, sulofenur, talisomycin, tecogalan sodium, tegafur, teloxantrone hydrochloride, temoporfin, teniposide, teroxirone, testolactone, thiamiprine, thioguanine, thiotepa, tiazofurin, tirapazamine, toremifene citrate, trestolone acetate, triciribine phosphate, trimetrexate, trimetrexate glucuronate, triptorelin, tubulozole hydrochloride, uracil mustard, uredepa, vapreotide, verteporfin, vinblastine sulfate, vincristine sulfate, vindesine, vindesine sulfate, vinepidine sulfate, vinglycinate sulfate, vinleurosine sulfate, vinorelbine tartrate, vinrosidine sulfate, vinzolidine sulfate, vorozole, zeniplatin, zinostatin, zorubicin hydrochloride, or a combination thereof.

V. Administration of Compositions and Activities

Various embodiments of the present disclosure provide a method of treating, reducing the severity of, and/or slowing the progression of a cancer, including one or more solid tumors, in an individual, wherein the individual is in need of cancer therapy. The method may comprise the steps of: administering at least one immunogenic composition, which may immunize the individual, such as by activating effector cells to expand in vivo; administrating a therapeutically effective amount of cells, including fibroblasts (which may be antigen presenting cells), or any other antigen presenting cells (such as dendritic cells), into the local tumor microenvironment; inducing tumor cell death (including immunogenic tumor cell death); administrating one or more compositions capable of augmenting antigen presentation in cancer cells; administrating one or more compositions, such as vaccines, capable of eliciting immunosurveillance to prevent tumor relapse, as well as to induce an abscopal effect; or a combination thereof. The steps may be performed once or multiple times, in any order. The steps may be performed concurrently or independently, or a combination of concurrently and independently.

In some embodiments, at least one immunogenic composition is administered at least once to an individual followed by at least one administration of a therapeutically effective amount of any cell encompassed herein (including any fibroblast encompassed herein) to the individual. In some embodiments, at least one immunogenic composition is administered at least once to an individual followed by at least one administration of a therapeutically effective amount of any cell encompassed herein (including any fibroblast encompassed herein) to the individual followed by at least one administration of at least one immunogenic composition.

In some embodiments, a therapeutically effective amount of any cell encompassed herein (including any fibroblast encompassed herein) is administered at least once to an individual followed by at least one administration of at least one immunogenic composition to the individual. In some embodiments, a therapeutically effective amount of any cell encompassed herein (including any fibroblast encompassed herein) is administered at least once to an individual followed by at least one administration of at least one immunogenic composition to the individual followed by administration of a therapeutically effective amount of any cell encompassed herein (including any fibroblast encompassed herein) to the individual.

In any embodiment, at least one composition that augments (or is capable of augmenting) antigen presentation may be given to an individual encompassed herein at any point relative to the administration of at least one immunogenic composition and/or any point relative to the administration of a therapeutically effective amount of any cell encompassed herein. In any embodiment, at least one composition and/or action that induces (or is capable of inducing) cancer cell death may be given to an individual encompassed herein at any point relative to the administration of at least one immunogenic composition and/or any point relative to the administration of a therapeutically effective amount of any cell encompassed herein.

In some embodiments, dendritic cells may be administered together with fibroblasts in a manner in which the fibroblasts attract and/or retain dendritic cells. In various embodiments, the immune cells, such as NK cells, T cells, NKT cells and/or gamma delta cells, are primed against a tumor cell lysate, tumor cell antigen, tumor cell cytokine, and/or stem cell lysate. In particular embodiments, the immunization means comprises treatment of the endogenous tumor with fibroblasts. Fibroblasts may be transfected to express one or more tumor immunogenic markers. In some embodiments, fibroblasts are grown and cultured together with allogeneic or autologous tumor cells. In some embodiments, hybridomas are generated comprising fibroblasts and tumor cells. In some embodiments, a fibroblast cell line is generated that expresses high antigenicity potential. In some embodiments, the fibroblast cell line is transfected with antigens to which immunity already exists in a patient. One example of such an antigen is the influenza derived peptides. Accordingly, in this example the fibroblast expressing flu peptides may be administered intratumorally and/or peritumorally with the aim of inducing an immune response that lyses the fibroblast and subsequently results in cross immunity to the tumor. In particular embodiments, “immunogenic fibroblasts” are fused with autologous tumor-derived cell lines.

In some embodiments, an immunogenic composition, including any immunogenic composition described herein, is administered to an individual, including an individual of the present disclosure. The immunogenic composition may be administered prior to cytotoxic and/or immunogenic cell death induction of the tumor. Immunization of the individual may be performed using known means in the art, including using suitable adjuvant(s). Assessment of immunity is performed by quantifying reactivity of T cells or B cells in response to protein antigens or derivatives thereof, derivatives including peptide antigens or other antigenic epitopes.

Responses may be assessed in terms of proliferative responses, cytokine release, antibody responses, or generation of cytotoxic T cells. Methods of assessing the responses are well known in the art. In at least one embodiment, antibody responses are assessed to a panel of tumor associated proteins subsequent to immunization of patient. Antibody responses are utilized to guide that peptides will be utilized for prior immunization. For example, if an individual is immunized with tumor antigen(s) on a weekly basis, the subsequent assessment of antibody responses is performed at approximately 1-3 months after initiation of immunization. Protocols for immunization include weekly, biweekly, or monthly immunization regimens. Assessment of antibody responses is performed utilizing standard enzyme linked immunosorbent (ELISA) assay or similar assays for detecting antibody responses. Assessment of antibodies is performed, in one embodiment of the disclosure, against proteins associated with tumor. In some embodiments, the immunogenic composition is a polyvalent vaccine, which is administered subsequent to administration of immunogenic fibroblasts.

Administration of tumor and/or pulsed dendritic cells may be utilized as a polyvalent vaccine, whereas subsequent to administration antibody or T cell responses are assessed for induction of antigen specificity, peptides corresponding to immune response stimulated are used for further immunization to focus the immune response.

The polypeptide and nucleic acid compositions disclosed herein may be administered to an individual, including an animal, such as a human, by a number of methods known in the art. Examples of suitable methods include intramuscular, intradermal, intraepidermal, intravenous, intraarterial, subcutaneous, or intraperitoneal administration, oral administration, and/or topical application (such as ocular, intranasal, and intravaginal application). One particular method of intradermal or intraepidermal administration of a nucleic acid vaccine composition that may be used is gene gun delivery using the Particle Mediated Epidermal Delivery (PMED™) vaccine delivery device marketed by PowderMed [35]. PMED™ is a needle-free method of administering vaccines to animals or humans. The PMED™ system involves the precipitation of DNA onto microscopic gold particles that are then propelled by helium gas into the epidermis [36]. The DNA-coated gold particles are delivered to the APCs and keratinocytes of the epidermis, and once inside the nuclei of these cells, the DNA elutes off the gold and becomes transcriptionally active, producing encoded protein. This protein is then presented by the APCs to the lymphocytes to induce a T-cell-mediated immune response.

Another particular method for intramuscular administration of a nucleic acid immunogenic composition (such as a vaccine) provided by the present disclosure is electroporation [37]. Electroporation uses controlled electrical pulses to create temporary pores in the cell membrane, which facilitates cellular uptake of the nucleic acid vaccine injected into the muscle [38-41]. Where a CpG is used in combination with a nucleic acid vaccine, it is preferred that the CpG and nucleic acid vaccine are co-formulated in one formulation and the formulation is administered intramuscularly by electroporation. A helper T cell and cytotoxic T cell stimulatory polypeptide can be introduced into a mammalian host, including humans, linked to its own carrier or as a homopolymer or heteropolymer of active polypeptide units. Such a polymer can elicit increase immunological reaction and, where different polypeptides are used to make up the polymer, the additional ability to induce antibodies and/or T cells that react with different antigenic determinants of the tumor. Useful carriers known in the art include, for example, thyroglobulin, albumins such as human serum albumin, tetanus toxoid, polyamino acids such as poly(D-lysine:D-glutamic acid), influenza polypeptide, and the like. Adjuvants such as incomplete Freunds adjuvant, GM-CSF, aluminum phosphate, CpG containing DNA, inulin, Poly (IC), aluminum hydroxide, alum, or montanide can also be used in the administration of an helper T cell and cytotoxic T cell stimulatory polypeptide.

In particular embodiments of the disclosure, a state of lymphopenia is induced in an individual, including an individual of the present disclosure. The lymphopenia may be induced prior to the administration of composition and/or actions disclosed herein. Any method known in the art may be used to induce lymphopenia, including but not limited to irradiation (including total lymphoid irradiation), administration of cyclophosphamide, or both. The induction of lymphopenia may cause homeostatic expansion of lymphocytes, which may reduce the costimulatory activation threshold by 10%, 20%, 30%, 40%, 50%, 60%, 70%, 80%, 90%, 100%, or more. The induction of lymphopenia may cause homeostatic proliferation of endogenous lymphocyte.

In some embodiments, cells of the present disclosure, including lymphocytes, may have an increase propensity for activation induced by a lymphocyte mitogen. The lymphocyte mitogen may comprise an interleukin treatment, including a interleukin-2 treatment, including a interleukin-7 treatment, including a interleukin-15 treatment, or a combination thereof.

In some embodiments, an immune-depressing composition is administered to an individual, including an individual of the present disclosure. The immune-depressing composition may be a phosphodiesterase (PDE)-5 inhibitor, such as acetildenafi, aildenafil, avanafil, benzamidenafil, homosildenafil, icariin, lodenafil, mirodenafil, nitrosoprodenafil, sildenafil, sulfoaildenafil, tadalafil, udenafil, vardenafil, zaprinast, or a combination thereof.

The individual of the present disclosure may have any type of cancer, including a brain tumor. The brain tumor may be, for example, a glioblastoma, a glioblastoma multiforme, an oligodendroglioma, a primitive neuroectodermal tumor, an astrocytoma, an ependymoma, an oligodendroglioma, a medulloblastoma, a meningioma, a pituitary carcinoma, a neuroblastoma, a craniopharyngioma, or a combination thereof.

In some embodiments of the disclosure, administration of intravenous vitamin C is utilized. Patients treated with immunotherapy have been shown to develop a scurvy-like condition. The patient presented with acute signs and symptoms of scurvy (perifollicular petechiae, erythema, gingivitis and bleeding). Serum ascorbate levels were significantly reduced to almost undetectable levels [283]. Although the role of ascorbic acid (AA) hyper-supplementation in stimulation of immunity in healthy subjects is controversial, it is well established that AA deficiency is associated with impaired cell mediated immunity. This has been demonstrated in numerous studies showing deficiency suppresses T cytotoxic responses, delayed type hypersensitivity, and bacterial clearance [284]. Additionally, it is well-known that NK activity, which IL-2 is anti-tumor activity is highly dependent on, is suppressed during conditions of AA deficiency [285]. Thus it may be that while IL-2 therapy on the one hand is stimulating T and NK function, the systemic inflammatory syndrome-like effects of this treatment may actually be suppressed by induction of a negative feedback loop. Such a negative feedback loop with IL-2 therapy was successfully overcome by work using low dose histamine to inhibit IL-2 mediated immune suppression, which led to the “drug” Ceplene (histamine dichloride) receiving approval as an IL-2 adjuvant for treatment of AML [286].

The concept of AA deficiency subsequent to IL-2 therapy was reported previously by another group. Marcus et al evaluated 11 advanced cancer patients suffering from melanoma, renal cell carcinoma and colon cancer being on a 3 phase immunotherapeutic program consisting of: a) 5 days of i.v. high-dose (10(5) units/kg every 8 h) interleukin 2, (b) 6½ days of rest plus leukapheresis; and (c) 4 days of high-dose interleukin 2 plus three infusions of autologous lymphokine-activated killer cells. Mean plasma ascorbic acid levels were normal (0.64+/−0.25 mg/dl) before therapy. Mean levels dropped by 80% after the first phase of treatment with high-dose interleukin 2 alone (0.13+/−0.08 mg/dl). Subsequently plasma ascorbic acid levels remained severely depleted (0.08 to 0.13 mg/dl) throughout the remainder of the treatment, becoming undetectable (less than 0.05 mg/dl) in eight of 11 patients during this time. Importantly, blood pantothenate and plasma vitamin E remained within normal limits in all 11 patients throughout the phases of therapy, suggesting the hypovitaminosis was specific AA. Strikingly, Responders (n=3) differed from nonresponders (n=8) in that plasma ascorbate levels in the former recovered to at least 0.1 mg/dl (frank clinical scurvy) during Phases 2 and 3, whereas levels in the latter fell below this level [287]. Similar results were reported in another study by the same group examining an additional 15 patients [288]. The possibility that prognosis was related to AA levels is intriguing because of the possibility of higher immune response in these patients, however this has not been tested.

The state of AA deficiency in cancer patients, whether or not as a result of inflammation, suggests supplementation may yield benefit in quality of life. Indeed this was one of the main findings that stimulating us to write this review [289]. Improvements in quality of life were also noted in the early studies of Murata et al [290], as well as Cameron [291]. But in addition to this endpoint there appears to be a growing number of studies suggesting direct anticancer effects via generation of free radicals locally at tumor sites [292]. In vitro studies on a variety of cancer cells including neuroblastoma [293], bladder cancer [294], pancreatic cancer [295], mesothelioma [296], hepatoma [297], have demonstrated cytotoxic effects at pharmacologically achievable concentrations.

Enhancement of cytotoxicity of Docetaxel, Epirubicin, Irinotecan and 5-FU to a battery of tumor cell lines by AA was demonstrated in vitro [298]. In vivo studies have also supported the potential anticancer effects of AA. For example, Pollard et al used the rat PAIII androgen-independent syngeneic prostate cancer cell line to induce tumors in Lobund-Wistar rats. Daily intraperitoneal administration of AA for 30 days, with evaluation at day 40 revealed significant inhibition of tumor growth, as well as reduction in pulmonary and lymphatic metastasis [299]. Levine's group reported successful in vivo inhibition of human xenografted glioma, ovarian, and neuroblastoma cells in immune deficient animals by administration of AA. Interestingly control fibroblasts were not affected [300]. Clinical reports of remission induced by IV AA have been published [301], however, as mentioned above, formal trials are still ongoing.

In addition to direct cytotoxicity of AA on tumor cells, inhibition of angiogenesis may be another mechanism of action. It has been reported that AA inhibits HUVEC proliferation in vitro [302], as well as suppressing neovascularization in the chorionic allontoic membrane assay [303]. Recently we have reported that in vivo administration of AA results in suppressed vascular cord formation in mouse models [304]. Supporting this possibility, Yeom et al demonstrated that parenteral administration of AA in the S-180 sarcoma model leads to reduced tumor growth, which was associated with suppression of angiogenesis and the pro-angiogenic factors bFGF, VEGF, and MMP-2 [305]. Recent studies suggest that AA suppresses activation of the hypoxia inducible factor (HIF)-1, which is a critical transcription factor that stimulates tumor angiogenesis [306-308]. The clinical relevance of this has been demonstrated in a study showing that endometrial cancer patients having reduced tumor ascorbate levels possess higher levels of HIF-1 activation and a more aggressive phenotype [309].

Thus the possibility exists that administration of AA for treatment of tumor inflammatory mediated pathologies may also cause an antitumor effect. Whether this effect is mediated by direct tumor cytotoxicity or inhibition of angiogenesis remains to be determined. Unfortunately none of the ongoing trials of AA in cancer patients seek to address this issue [310-315].

Despite numerous claims in the popular media and even on vitamin labels, the concept of AA stimulating immunity is not as clear-cut. Part of this is because ROS are involved in numerous signals of immune cells [316]. For example, it is known that T cell receptor signaling induces an intracellular flux of ROS which is necessary for T cell activation [317]. There are numerous studies demonstrating ascorbic acid under certain conditions actually can inhibit immunity. For example, high dose ascorbate inhibits T cell and B cell proliferative responses as well as IL-2 secretion in vitro [318, 319], as well as NK cytotoxic activity [320]. Additionally, AA has been demonstrated to inhibit T cell activating ability of dendritic cells by rendering them in an immature state in part through inhibition of NF-kappa B [321].

However, it appears that the immune stimulatory effects of AA are actually observed in the context of background immune suppression or in situations of AA deficiency, both of which are well-known in the cancer and SIRS patient. A common occurrence in cancer [322-326] and SIRS patients [327, 328] is the presence of a cleaved T cell receptor (TCR) zeta chain. The zeta chain is an important component of T cell and NK cell activation, that bears the highest number of immunoreceptor tyrosine-based activation motifs (ITAMs) of other TCR and NK signaling molecules [329]. At a cellular level cleavage of the zeta chain is associated with loss of T/NK cell function and spontaneous apoptosis [330-332], at a clinical level it is associated with poor prognosis [333-338].

Since loss of TCR zeta chain is found in other inflammatory conditions ranging from hemodialysis [339, 340], to autoimmunity [341-344], to heart disease [345], the possibility that inflammatory mediators such as ROS cause TCR zeta downregulation has been suggested. Circumstantial evidence comes from studies associated inflammatory cells such as tumor associated macrophages (TAMS) with suppression of zeta chain expression [346]. Myeloid suppressor cells, which are known to produce high concentrations of ROS [347-349] have also been demonstrated to induce reduction of TCR zeta chain in cancer [350], and post trauma [351]. Administration of anti-oxidants has been shown to reverse TCR zeta chain cleavage in tissue culture [352, 353]. Therefore, from the T cell side of immunity, an argument could be made that intravenous ascorbic acid may upregulate immunity by blocking zeta chain downregulation in the context of cancer and acute inflammation.

While it is known that AA functions as an antioxidant in numerous biological conditions, as well as reduces inflammatory markers, the possibility that AA actually increases immune function in cancer patients, as well as is effects on survival and other cancer-related events, has never been formally tested. IV AA has a long and controversial history in relation to reducing tumors in patients. This has impeded research into other potential benefits of this therapy in cancer patients such as reduction of inflammation, improvement of quality of life, and impeding SIRS initiation and progression to MOF. While ongoing clinical trials of IV AA for cancer may or may not meet the bar to grant this modality a place amongst the recognized chemotherapeutic agents, it is critical that we collect as much biological data as possible, given the possibility of this agent to be a meaningful adjuvant therapy.

In some embodiments of the disclosure, administration of AA together with fibroblasts is performed to enhance anticancer activities of fibroblasts.

It is to be understood that the embodiments of the application disclosed herein are illustrative of the principles of the embodiments of the application. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

Various embodiments of the disclosure are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the disclosure known to the applicant at this time of filing the application has been presented and is intended for the purposes of illustration and description. The present description is not intended to be exhaustive nor limit the disclosure to the precise form disclosed and many modifications and variations are possible in the light of the above teachings. The embodiments described serve to explain the principles of the disclosure and its practical application and to enable others skilled in the art to utilize the disclosure in various embodiments and with various modifications as are suited to the particular use contemplated. Therefore, it is intended that the disclosure not be limited to the particular embodiments disclosed for carrying out the disclosure.

While particular embodiments of the present disclosure have been shown and described, it will be obvious to those skilled in the art that, based upon the teachings herein, changes and modifications may be made without departing from this disclosure and its broader aspects and, therefore, the appended claims are to encompass within their scope all such changes and modifications as are within the true spirit and scope of this disclosure.

Although the present disclosure and its advantages have been described in detail, it should be understood that various changes, substitutions and alterations can be made herein without departing from the spirit and scope of the design as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the present disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

### REFERENCES

All publications and works of art discussed or cited herein are incorporated by reference in their entirety to the same extent as if each individual publication or patent application was specifically and individually indicated to be incorporated by reference. The following description includes information that may be useful in understanding the present disclosure. It is not an admission that any of the information provided herein is prior art or relevant to the presently claimed disclosure, or that any publication specifically or implicitly referenced is prior art.
1. Coley, W. B., The treatment of malignant tumors by repeated inoculations of erysipelas. With a report of ten original cases. 1893. Clin Orthop Relat Res, 1991(262): p. 3-11.
2. Coley, W. B., The Treatment of Sarcoma of the Long Bones. Ann Surg, 1933. 97(3): p. 434-60.
3. McCarthy, E. F., The toxins of William B. Coley and the treatment of bone and soft-tissue sarcomas. Iowa Orthop J, 2006. 26: p. 154-8.
4. Bickels, J., et al., Coley's toxin: historical perspective. Isr Med Assoc J, 2002. 4(6): p. 471-2.
5. Wiemann, B. and C. O. Starnes, Coley's toxins, tumor necrosis factor and cancer research: a historical perspective. Pharmacol Ther, 1994. 64(3): p. 529-64.
6. Starnes, C. O., Coley's toxins. Nature, 1992. 360(6399): p. 23.
7. Brouckaert, P. G., W. Fiers, and F. J. Lejeune, Coley's vaccine and TNF therapy. Nature, 1992. 358(6388): p. 630.
8. Nauts, H. C., G. A. Fowler, and F. H. Bogatko, A review of the influence of bacterial infection and of bacterial products (Coley's toxins) on malignant tumors in man; a critical analysis of 30 inoperable cases treated by Coley's mixed toxins, in which diagnosis was confirmed by microscopic examination selected for special study. Acta Med Scand Suppl, 1953. 276: p. 1-103.
9. Brower, V., Approval of provenge seen as first step for cancer treatment vaccines. J Natl Cancer Inst, 2010. 102(15): p. 1108-10.
10. Lipson, E. J. and C. G. Drake, Ipilimumab: an anti-CTLA-4 antibody for metastatic melanoma. Clin Cancer Res, 2011. 17(22): p. 6958-62.
11. Rajakulendran, T. and D. N. Adam, Spotlight on pembrolizumab in the treatment of advanced melanoma. Drug Des Devel Ther, 2015. 9: p. 2883-6.
12. Raedler, L. A., Opdivo (Nivolumab): Second PD-1 Inhibitor Receives FDA Approval for Unresectable or Metastatic Melanoma. Am Health Drug Benefits, 2015. 8(Spec Feature): p. 180-3.
13. Sweis, R. F. and J. J. Luke, Mechanistic and pharmacologic insights on immune checkpoint inhibitors. Pharmacol Res, 2017. 120: p. 1-9.
14. Martin-Fontecha, A., et al., Induced recruitment of NK cells to lymph nodes provides IFN-gamma for T(H)1 priming. Nat Immunol, 2004. 5(12): p. 1260-5.
15. Morandi, B., et al., NK cells of human secondary lymphoid tissues enhance T cell polarization via IFN-gamma secretion. Eur J Immunol, 2006. 36(9): p. 2394-400.
16. Ksienzyk, A., et al., IRF-1 expression is essential for natural killer cells to suppress metastasis. Cancer Res, 2011. 71(20): p. 6410-8.
17. Lopez-Soto, A., et al., Control of Metastasis by NK Cells. Cancer Cell, 2017. 32(2): p. 135-154.
18. Krasnova, Y., et al., Bench to bedside: NK cells and control of metastasis. Clin Immunol, 2017. 177: p. 50-59.
19. Putz, E. M., et al., NK cell heparanase controls tumor invasion and immune surveillance. J Clin Invest, 2017. 127(7): p. 2777-2788.
20. Morvan, M. G. and L. L. Lanier, NK cells and cancer: you can teach innate cells new tricks. Nat Rev Cancer, 2016. 16(1): p. 7-19.
21. Maccalli, C., et al., Soluble NKG2D ligands are biomarkers associated with the clinical outcome to immune checkpoint blockade therapy of metastatic melanoma patients. Oncoimmunology, 2017. 6(7): p. e1323618.
22. Goding, S. R., et al., Adoptive transfer of natural killer cells promotes the anti-tumor efficacy of T cells. Clin Immunol, 2017. 177: p. 76-86.
23. Muraro, E., et al., Improved Natural Killer cell activity and retained anti-tumor CD8(+) T cell responses contribute to the induction of a pathological complete response in HER2-positive breast cancer patients undergoing neoadjuvant chemotherapy. J Transl Med, 2015. 13: p. 204.
24. Lee, S. C., et al., Natural killer (NK): dendritic cell (DC) cross talk induced by therapeutic monoclonal antibody triggers tumor antigen-specific T cell immunity. Immunol Res, 2011. 50(2-3): p. 248-54.
25. Beano, A., et al., Correlation between NK function and response to trastuzumab in metastatic breast cancer patients. J Transl Med, 2008. 6: p. 25.
26. Jaime-Ramirez, A. C., et al., IL-12 enhances the antitumor actions of trastuzumab via NK cell IFN-gamma production. J Immunol, 2011. 186(6): p. 3401-9.
27. Charlebois, R., et al., PolyI:C and CpG Synergize with Anti-ErbB2 mAb for Treatment of Breast Tumors Resistant to Immune Checkpoint Inhibitors. Cancer Res, 2017. 77(2): p. 312-319.
28. Hoshimoto, S., et al., Assessment of prognostic circulating tumor cells in a phase III trial of adjuvant immunotherapy after complete resection of stage IV melanoma. Ann Surg, 2012. 255(2): p. 357-62.
29. Kalani, A. D., et al., Immunotherapy as an adjuvant therapy in the management of advanced, surgically resected, melanoma. G Ital Dermatol Venereol, 2008. 143(1): p. 59-70.
30. Ugel, S., et al., Targeting tumor vasculature: expanding the potential of DNA cancer vaccines. Cancer Immunol Immunother, 2015. 64(10): p. 1339-48.
31. Li, L., et al., Developing a clinical development paradigm for translation of a mammaglobin—A DNA vaccine. Immunotherapy, 2015: p. 1-3.
32. Tiriveedhi, V., et al., Safety and preliminary evidence of biologic efficacy of a mammaglobin—a DNA vaccine in patients with stable metastatic breast cancer. Clin Cancer Res, 2014. 20(23): p. 5964-75.
33. Heller, R. and L. C. Heller, Gene electrotransfer clinical trials. Adv Genet, 2015. 89: p. 235-62.
34. Butterfield, L. H., et al., Alpha fetoprotein DNA prime and adenovirus boost immunization of two hepatocellular cancer patients. J Transl Med, 2014. 12: p. 86.
35. Sharpe, M., et al., Protection of mice from H5N1 influenza challenge by prophylactic DNA vaccination using particle mediated epidermal delivery. Vaccine, 2007. 25(34): p. 6392-8.
36. Loudon, P. T., et al., GM-CSF increases mucosal and systemic immunogenicity of an H1N1 influenza DNA vaccine administered into the epidermis of non-human primates. PLoS One, 2010. 5(6): p. e11021.
37. Jones, S., et al., DNA vaccination protects against an influenza challenge in a double-blind randomised placebo-controlled phase 1b clinical trial. Vaccine, 2009. 27(18): p. 2506-12.
38. Shah, M. A., et al., DNA Mediated Vaccines Delivery Through Nanoparticles. J Nanosci Nanotechnol, 2015. 15(1): p. 41-53.
39. Mpendo, J., et al., A Phase I Double Blind, Placebo-Controlled, Randomized Study of the Safety and Immunogenicity of Electroporated HIV DNA with or without Interleukin 12 in Prime-Boost Combinations with an Ad35 HIV Vaccine in Healthy HIV-Seronegative African Adults. PLoS One, 2015. 10(8): p. e0134287.
40. Keane-Myers, A. M., et al., DNA electroporation of multi-agent vaccines conferring protection against select agent challenge: TriGrid delivery system. Methods Mol Biol, 2014. 1121: p. 325-36.
41. Hooper, J. W., et al., A Phase 1 clinical trial of Hantaan virus and Puumala virus M—segment DNA vaccines for haemorrhagic fever with renal syndrome delivered by intramuscular electroporation. Clin Microbiol Infect, 2014. 20 Suppl 5: p. 110-7.
42. van Furth, R. and Z. A. Cohn, The origin and kinetics of mononuclear phagocytes. J Exp Med, 1968. 128(3): p. 415-35.
43. Wynn, T. A., A. Chawla, and J. W. Pollard, Macrophage biology in development, homeostasis and disease. Nature, 2013. 496(7446): p. 445-55.
44. Smith, T. D., et al., Harnessing macrophage plasticity for tissue regeneration. Adv Drug Deliv Rev, 2017.
45. Vannella, K. M. and T. A. Wynn, Mechanisms of Organ Injury and Repair by Macrophages. Annu Rev Physiol, 2017. 79: p. 593-617.
46. Boddupalli, A., L. Zhu, and K. M. Bratlie, Methods for Implant Acceptance and Wound Healing: Material Selection and Implant Location Modulate Macrophage and Fibroblast Phenotypes. Adv Healthc Mater, 2016. 5(20): p. 2575-2594.
47. Snyder, R. J., et al., Macrophages: A review of their role in wound healing and their therapeutic use. Wound Repair Regen, 2016. 24(4): p. 613-29.
48. Gombozhapova, A., et al., Macrophage activation and polarization in post-infarction cardiac remodeling. J Biomed Sci, 2017. 24(1): p. 13.
49. Hu, Y., et al., Class A scavenger receptor attenuates myocardial infarction-induced cardiomyocyte necrosis through suppressing M1 macrophage subset polarization. Basic Res Cardiol, 2011. 106(6): p. 1311-28.
50. Ma, Y., et al., Matrix metalloproteinase-28 deletion exacerbates cardiac dysfunction and rupture after myocardial infarction in mice by inhibiting M2 macrophage activation. Circ Res, 2013. 112(4): p. 675-88.
51. Lee, C. W., et al., Macrophage heterogeneity of culprit coronary plaques in patients with acute myocardial infarction or stable angina. Am J Clin Pathol, 2013. 139(3): p. 317-22.
52. Yan, X., et al., Temporal dynamics of cardiac immune cell accumulation following acute myocardial infarction. J Mol Cell Cardiol, 2013. 62: p. 24-35.
53. Fernandez-Velasco, M., S. Gonzalez-Ramos, and L. Bosca, Involvement of monocytes/macrophages as key factors in the development and progression of cardiovascular diseases. Biochem J, 2014. 458(2): p. 187-93.
54. de Couto, G., et al., Macrophages mediate cardioprotective cellular postconditioning in acute myocardial infarction. J Clin Invest, 2015. 125(8): p. 3147-62.
55. Guiteras, R., M. Flaquer, and J. M. Cruzado, Macrophage in chronic kidney disease. Clin Kidney J, 2016. 9(6): p. 765-771.
56. Meng, X. M., et al., Macrophage Phenotype in Kidney Injury and Repair. Kidney Dis (Basel), 2015. 1(2): p. 138-46.
57. Yamamoto, S., et al., Atherosclerosis following renal injury is ameliorated by pioglitazone and losartan via macrophage phenotype. Atherosclerosis, 2015. 242(1): p. 56-64.
58. Li, C., et al., Enhanced M1 and Impaired M2 Macrophage Polarization and Reduced Mitochondrial Biogenesis via Inhibition of AMP Kinase in Chronic Kidney Disease. Cell Physiol Biochem, 2015. 36(1): p. 358-72.
59. Sun, Y. Y., et al., Macrophage Phenotype in Liver Injury and Repair. Scand J Immunol, 2017. 85(3): p. 166-174.
60. Gratchev, A., et al., Mphi1 and Mphi2 can be re-polarized by Th2 or Th1 cytokines, respectively, and respond to exogenous danger signals. Immunobiology, 2006. 211(6-8): p. 473-86.
61. Mills, C. D., M1 and M2 Macrophages: Oracles of Health and Disease. Crit Rev Immunol, 2012. 32(6): p. 463-88.
62. Mills, C. D. and K. Ley, M1 and M2 macrophages: the chicken and the egg of immunity. J Innate Immun, 2014. 6(6): p. 716-26.
63. Alsaid, H., et al., Non invasive imaging assessment of the biodistribution of GSK2849330, an ADCC and CDC optimized anti HER3 mAb, and its role in tumor macrophage recruitment in human tumor-bearing mice. PLoS One, 2017. 12(4): p. e0176075.
64. Josephs, D. H., et al., Anti-Folate Receptor-alpha IgE but not IgG Recruits Macrophages to Attack Tumors via TNFalpha/MCP-1 Signaling. Cancer Res, 2017. 77(5): p. 1127-1141.
65. Velmurugan, R., et al., Macrophage-Mediated Trogocytosis Leads to Death of Antibody-Opsonized Tumor Cells. Mol Cancer Ther, 2016. 15(8): p. 1879-89.
66. Gul, N. and M. van Egmond, Antibody-Dependent Phagocytosis of Tumor Cells by Macrophages: A Potent Effector Mechanism of Monoclonal Antibody Therapy of Cancer. Cancer Res, 2015. 75(23): p. 5008-13.
67. Church, A. K., et al., Anti-CD20 monoclonal antibody-dependent phagocytosis of chronic lymphocytic leukaemia cells by autologous macrophages. Clin Exp Immunol, 2016. 183(1): p. 90-101.
68. Shi, Y., et al., Trastuzumab triggers phagocytic killing of high HER2 cancer cells in vitro and in vivo by interaction with Fcgamma receptors on macrophages. J Immunol, 2015. 194(9): p. 4379-86.
69. Weiskopf, K. and I. L. Weissman, Macrophages are critical effectors of antibody therapies for cancer. MAbs, 2015. 7(2): p. 303-10.
70. Oflazoglu, E., et al., Macrophages contribute to the antitumor activity of the anti-CD30 antibody SGN-30. Blood, 2007. 110(13): p. 4370-2.
71. Osman, R., et al., Calreticulin Release at an Early Stage of Death Modulates the Clearance by Macrophages of Apoptotic Cells. Front Immunol, 2017. 8: p. 1034.
72. Feng, M., et al., Macrophages eat cancer cells using their own calreticulin as a guide: roles of TLR and Btk. Proc Natl Acad Sci USA, 2015. 112(7): p. 2145-50.
73. Chao, M. P., et al., Calreticulin is the dominant pro-phagocytic signal on multiple human cancers and is counterbalanced by CD47. Sci Transl Med, 2010. 2(63): p. 63ra94.
74. Murata, Y., et al., The CD47-SIRPalpha signalling system: its physiological roles and therapeutic application. J Biochem, 2014. 155(6): p. 335-44.
75. Roberts, D. D., S. Kaur, and D. R. Soto-Pantoja, Therapeutic targeting of the thrombospondin-1 receptor CD47 to treat liver cancer. J Cell Commun Signal, 2015. 9(1): p. 101-2.
76. Liu, J., et al., Pre-Clinical Development of a Humanized Anti-CD47 Antibody with Anti-Cancer Therapeutic Potential. PLoS One, 2015. 10(9): p. e0137345.
77. Weiskopf, K., et al., CD47-blocking immunotherapies stimulate macrophage-mediated destruction of small-cell lung cancer. J Clin Invest, 2016. 126(7): p. 2610-20.
78. Weiskopf, K., et al., Eradication of Canine Diffuse Large B-Cell Lymphoma in a Murine Xenograft Model with CD47 Blockade and Anti-CD20. Cancer Immunol Res, 2016. 4(12): p. 1072-1087.
79. Zeng, D., et al., A fully human anti-CD47 blocking antibody with therapeutic potential for cancer. Oncotarget, 2016. 7(50): p. 83040-83050.
80. Liljefors, M., et al., Influence of varying doses of granulocyte-macrophage colony-stimulating factor on pharmacokinetics and antibody-dependent cellular cytotoxicity. Cancer Immunol Immunother, 2008. 57(3): p. 379-88.
81. Tarr, P. E., Granulocyte-macrophage colony-stimulating factor and the immune system. Med Oncol, 1996. 13(3): p. 133-40.
82. Ragnhammar, P., et al., Cytotoxicity of white blood cells activated by granulocyte-colony-stimulating factor, granulocyte/macrophage-colony-stimulating factor and macrophage-colony-stimulating factor against tumor cells in the presence of various monoclonal antibodies. Cancer Immunol Immunother, 1994. 39(4): p. 254-62.
83. Ragnhammar, P., Anti-tumoral effect of GM-CSF with or without cytokines and monoclonal antibodies in solid tumors. Med Oncol, 1996. 13(3): p. 167-76.
84. Lin, E. Y., et al., Colony-stimulating factor 1 promotes progression of mammary tumors to malignancy. J Exp Med, 2001. 193(6): p. 727-40.
85. Aharinejad, S., et al., Colony-stimulating factor-1 blockade by antisense oligonucleotides and small interfering RNAs suppresses growth of human mammary tumor xenografts in mice. Cancer Res, 2004. 64(15): p. 5378-84.
86. Lin, E. Y., et al., Macrophages regulate the angiogenic switch in a mouse model of breast cancer. Cancer Res, 2006. 66(23): p. 11238-46.
87. Lin, E. Y. and J. W. Pollard, Tumor-associated macrophages press the angiogenic switch in breast cancer. Cancer Res, 2007. 67(11): p. 5064-6.
88. Zhang, W. J., et al., Hypoxia-inducible factor-1 alpha Correlates with Tumor-Associated Macrophages Infiltration, Influences Survival of Gastric Cancer Patients. J Cancer, 2017. 8(10): p. 1818-1825.
89. Yuan, X., et al., Prognostic significance of tumor-associated macrophages in ovarian cancer: A meta-analysis. Gynecol Oncol, 2017. 147(1): p. 181-187.
90. Ma, C., et al., CD163-positive cancer cells are potentially associated with high malignant potential in clear cell renal cell carcinoma. Med Mol Morphol, 2017.
91. Shi, Y., et al., Tumour-associated macrophages secrete pleiotrophin to promote PTPRZ1 signalling in glioblastoma stem cells for tumour growth. Nat Commun, 2017. 8: p. 15080.
92. Zhao, X., et al., Prognostic significance of tumor-associated macrophages in breast cancer: a meta-analysis of the literature. Oncotarget, 2017. 8(18): p. 30576-30586.
93. Pearce, O. M., et al., Inverse hormesis of cancer growth mediated by narrow ranges of tumor-directed antibodies. Proc Natl Acad Sci USA, 2014. 111(16): p. 5998-6003.
94. Pander, J., et al., Activation of tumor-promoting type 2 macrophages by EGFR-targeting antibody cetuximab. Clin Cancer Res, 2011. 17(17): p. 5668-73.
95. Clynes, R. A., et al., Inhibitory Fc receptors modulate in vivo cytotoxicity against tumor targets. Nat Med, 2000. 6(4): p. 443-6.
96. Pricop, L., et al., Differential modulation of stimulatory and inhibitory Fc gamma receptors on human monocytes by Th1 and Th2 cytokines. J Immunol, 2001. 166(1): p. 531-7.
97. Tridandapani, S., et al., Regulated expression and inhibitory function of Fcgamma RIM in human monocytic cells. J Biol Chem, 2002. 277(7): p. 5082-9.
98. Joshi, T., et al., Molecular analysis of expression and function of hFcgammaRIIb1 and b2 isoforms in myeloid cells. Mol Immunol, 2006. 43(7): p. 839-50.
99. Wijngaarden, S., et al., A shift in the balance of inhibitory and activating Fcgamma receptors on monocytes toward the inhibitory Fcgamma receptor IIb is associated with prevention of monocyte activation in rheumatoid arthritis. Arthritis Rheum, 2004. 50(12): p. 3878-87.
100. Butchar, J. P., et al., Reciprocal regulation of activating and inhibitory Fc[gamma] receptors by TLR7/8 activation: implications for tumor immunotherapy. Clin Cancer Res, 2010. 16(7): p. 2065-75.
101. Fatehchand, K., et al., Toll-like Receptor 4 Ligands Down-regulate Fcgamma Receptor IIb (FcgammaRIIb) via MARCH3 Protein-mediated Ubiquitination. J Biol Chem, 2016. 291(8): p. 3895-904.
102. Ghochikyan, A., et al., Targeting TLR-4 with a novel pharmaceutical grade plant derived agonist, Immunomax®, as a therapeutic strategy for metastatic breast cancer. J Transl Med, 2014. 12: p. 322.
103. Oronsky, B., et al., RRx-001: a systemically non-toxic M2-to-M1 macrophage stimulating and prosensitizing agent in Phase II clinical trials. Expert Opin Investig Drugs, 2017. 26(1): p. 109-119.
104. Lee, C., et al., Melittin suppresses tumor progression by regulating tumor-associated macrophages in a Lewis lung carcinoma mouse model. Oncotarget, 2017. 8(33): p. 54951-54965.
105. Zhang, Q., et al., Clinical Effects of CpG-Based Treatment on the Efficacy of Hepatocellular Carcinoma by Skewing Polarization Toward M1 Macrophage from M2. Cancer Biother Radiopharm, 2017. 32(6): p. 215-219.
106. Sato, T., et al., Intrapulmonary Delivery of CpG Microparticles Eliminates Lung Tumors. Mol Cancer Ther, 2015. 14(10): p. 2198-205.
107. Chiang, C. F., et al., Metformin-treated cancer cells modulate macrophage polarization through AMPK-NF-kappaB signaling. Oncotarget, 2017. 8(13): p. 20706-20718.
108. Kang, H., et al., Puerarin inhibits M2 polarization and metastasis of tumor-associated macrophages from NSCLC xenograft model via inactivating MEK/ERK 1/2 pathway. Int J Oncol, 2017. 50(2): p. 545-554.
109. Jia, X., et al., Emodin suppresses pulmonary metastasis of breast cancer accompanied with decreased macrophage recruitment and M2 polarization in the lungs. Breast Cancer Res Treat, 2014. 148(2): p. 291-302.
110. Xue, N., et al., Chlorogenic acid inhibits glioblastoma growth through repolarizating macrophage from M2 to M1 phenotype. Sci Rep, 2017. 7: p. 39011.
111. Sloan, E. K., et al., The sympathetic nervous system induces a metastatic switch in primary breast cancer. Cancer Res, 2010. 70(18): p. 7042-52.
112. Liu, B., et al., Polarization of M1 tumor associated macrophage promoted by the activation of TLR3 signal pathway. Asian Pac J Trop Med, 2016. 9(5): p. 484-8.
113. Liu, Q., et al., NMAAPJ Expressed in BCG-Activated Macrophage Promotes M1 Macrophage Polarization. Mol Cells, 2015. 38(10): p. 886-94.
114. Liu, Y., et al., Polysaccharide Agaricus blazei Murill stimulates myeloid derived suppressor cell differentiation from M2 to M1 type, which mediates inhibition of tumour immune-evasion via the Toll-like receptor 2 pathway. Immunology, 2015. 146(3): p. 379-91.
115. Yang, Y., et al., LPS converts Gr-1(+)CD115(+) myeloid-derived suppressor cells from M2 to M1 via P38 MAPK. Exp Cell Res, 2013. 319(12): p. 1774-83.
116. Sanchez-Quesada, C., A. Lopez-Biedma, and J. J. Gaforio, Maslinic Acid enhances signals for the recruitment of macrophages and their differentiation to m1 state. Evid Based Complement Alternat Med, 2015. 2015: p. 654721.
117. Dominguez-Soto, A., et al., Intravenous immunoglobulin promotes antitumor responses by modulating macrophage polarization. J Immunol, 2014. 193(10): p. 5181-9.
118. Yin, Y., et al., Phosphatidylserine-targeting antibody induces M1 macrophage polarization and promotes myeloid-derived suppressor cell differentiation. Cancer Immunol Res, 2013. 1(4): p. 256-68.
119. Deng, R., et al., Dimethyl Sulfoxide Suppresses Mouse 4T1 Breast Cancer Growth by Modulating Tumor-Associated Macrophage Differentiation. J Breast Cancer, 2014. 17(1): p. 25-32.
120. Mitsuhashi, A., et al., Surfactant protein A suppresses lung cancer progression by regulating the polarization of tumor-associated macrophages. Am J Pathol, 2013. 182(5): p. 1843-53.
121. Coscia, M., et al., Zoledronic acid repolarizes tumour-associated macrophages and inhibits mammary carcinogenesis by targeting the mevalonate pathway. J Cell Mol Med, 2010. 14(12): p. 2803-15.
122. Eriksson, F., et al., Tumor-specific bacteriophages induce tumor destruction through activation of tumor-associated macrophages. J Immunol, 2009. 182(5): p. 3105-11.
123. Steinman, R. M. and Z. A. Cohn, Identification of a novel cell type in peripheral lymphoid organs of mice. I. Morphology, quantitation, tissue distribution. J Exp Med, 1973. 137(5): p. 1142-62.
124. Banchereau, J. and R. M. Steinman, Dendritic cells and the control of immunity. Nature, 1998. 392(6673): p. 245-52.
125. Trombetta, E. S. and I. Mellman, Cell biology of antigen processing in vitro and in vivo. Annu Rev Immunol, 2005. 23: p. 975-1028.
126. Itano, A. A. and M. K. Jenkins, Antigen presentation to naive CD4 T cells in the lymph node. Nat Immunol, 2003. 4(8): p. 733-9.
127. Tjoa, B. A., et al., Evaluation of phase I/II clinical trials in prostate cancer with dendritic cells and PSMA peptides. Prostate, 1998. 36(1): p. 39-44.
128. Murphy, G. P., et al., Infusion of dendritic cells pulsed with HLA-A2-specific prostate-specific membrane antigen peptides: a phase II prostate cancer vaccine trial involving patients with hormone-refractory metastatic disease. Prostate, 1999. 38(1): p. 73-8.
129. Lodge, P. A., et al., Dendritic cell-based immunotherapy of prostate cancer: immune monitoring of a phase II clinical trial. Cancer Res, 2000. 60(4): p. 829-33.
130. Burch, P. A., et al., Priming tissue-specific cellular immunity in a phase I trial of autologous dendritic cells for prostate cancer. Clin Cancer Res, 2000. 6(6): p. 2175-82.
131. Nestle, F. O., et al., Vaccination of melanoma patients with peptide- or tumor lysate-pulsed dendritic cells. Nat Med, 1998. 4(3): p. 328-32.
132. Chakraborty, N. G., et al., Immunization with a tumor-cell-lysate-loaded autologous-antigen-presenting-cell-based vaccine in melanoma. Cancer Immunol Immunother, 1998. 47(1): p. 58-64.
133. Wang, F., et al., Phase I trial of a MART-1 peptide vaccine with incomplete Freund's adjuvant for resected high-risk melanoma. Clin Cancer Res, 1999. 5(10): p. 2756-65.
134. Thurner, B., et al., Vaccination with mage-3A1 peptide pulsed mature, monocyte-derived dendritic cells expands specific cytotoxic T cells and induces regression of some metastases in advanced stage IV melanoma. J Exp Med, 1999. 190(11): p. 1669-78.
135. Thomas, R., et al., Immature human monocyte-derived dendritic cells migrate rapidly to draining lymph nodes after intradermal injection for melanoma immunotherapy. Melanoma Res, 1999. 9(5): p. 474-81.
136. Mackensen, A., et al., Phase I study in melanoma patients of a vaccine with peptide pulsed dendritic cells generated in vitro from CD34(+) hematopoietic progenitor cells. Int J Cancer, 2000. 86(3): p. 385-92.
137. Panelli, M. C., et al., Phase 1 study in patients with metastatic melanoma of immunization with dendritic cells presenting epitopes derived from the melanoma-associated antigens MART-1 and gp100. J Immunother, 2000. 23(4): p. 487-98.
138. Schuler-Thurner, B., et al., Mage-3 and influenza-matrix peptide-specific cytotoxic T cells are inducible in terminal stage HLA-A2.1+ melanoma patients by mature monocyte-derived dendritic cells. J Immunol, 2000. 165(6): p. 3492-6.
139. Lau, R., et al., Phase I trial of intravenous peptide-pulsed dendritic cells in patients with metastatic melanoma. J Immunother, 2001. 24(1): p. 66-78.
140. Banchereau, J., et al., Immune and clinical responses in patients with metastatic melanoma to CD34(+) progenitor-derived dendritic cell vaccine. Cancer Res, 2001. 61(17): p. 6451-8.
141. Schuler-Thurner, B., et al., Rapid induction of tumor-specific type 1 T helper cells in metastatic melanoma patients by vaccination with mature, cryopreserved, peptide-loaded monocyte-derived dendritic cells. J Exp Med, 2002. 195(10): p. 1279-88.
142. Palucka, A. K., et al., Single injection of CD34+ progenitor-derived dendritic cell vaccine can lead to induction of T-cell immunity in patients with stage IV melanoma. J Immunother, 2003. 26(5): p. 432-9.
143. Bedrosian, I., et al., Intranodal administration of peptide pulsed mature dendritic cell vaccines results in superior CD8+ T-cell function in melanoma patients. J Clin Oncol, 2003. 21(20): p. 3826-35.
144. Slingluff, C. L., Jr., et al., Clinical and immunologic results of a randomized phase II trial of vaccination using four melanoma peptides either administered in granulocyte-macrophage colony-stimulating factor in adjuvant or pulsed on dendritic cells. J Clin Oncol, 2003. 21(21): p. 4016-26.
145. Hersey, P., et al., Phase I/II study of treatment with dendritic cell vaccines in patients with disseminated melanoma. Cancer Immunol Immunother, 2004. 53(2): p. 125-34.
146. Vilella, R., et al., Pilot study of treatment of biochemotherapy-refractory stage IV melanoma patients with autologous dendritic cells pulsed with a heterologous melanoma cell line lysate. Cancer Immunol Immunother, 2004. 53(7): p. 651-8.
147. Palucka, A. K., et al., Spontaneous proliferation and type 2 cytokine secretion by CD4+ T cells in patients with metastatic melanoma vaccinated with antigen pulsed dendritic cells. J Clin Immunol, 2005. 25(3): p. 288-95.
148. Banchereau, J., et al., Immune and clinical outcomes in patients with stage IV melanoma vaccinated with peptide-pulsed dendritic cells derived from CD34+ progenitors and activated with type I interferon. J Immunother, 2005. 28(5): p. 505-16.
149. Trakatelli, M., et al., A new dendritic cell vaccine generated with interleukin-3 and interferon-beta induces CD8+ T cell responses against NA17-A2 tumor peptide in melanoma patients. Cancer Immunol Immunother, 2006. 55(4): p. 469-74.
150. Salcedo, M., et al., Vaccination of melanoma patients using dendritic cells loaded with an allogeneic tumor cell lysate. Cancer Immunol Immunother, 2006. 55(7): p. 819-29.
151. Linette, G. P., et al., Immunization using autologous dendritic cells pulsed with the melanoma-associated antigen gp100-derived G280-9V peptide elicits CD8+ immunity. Clin Cancer Res, 2005. 11(21): p. 7692-9.
152. Escobar, A., et al., Dendritic cell immunizations alone or combined with low doses of interleukin-2 induce specific immune responses in melanoma patients. Clin Exp Immunol, 2005. 142(3): p. 555-68.
153. Tuettenberg, A., et al., Induction of strong and persistent MelanA/MART-1-specific immune responses by adjuvant dendritic cell-based vaccination of stage II melanoma patients. Int J Cancer, 2006. 118(10): p. 2617-27.
154. Schadendorf, D., et al., Dacarbazine (DTIC) versus vaccination with autologous peptide pulsed dendritic cells (DC) in first-line treatment of patients with metastatic melanoma: a randomized phase III trial of the DC study group of the DeCOG. Ann Oncol, 2006. 17(4): p. 563-70.
155. Di Pucchio, T., et al., Immunization of stage IV melanoma patients with Melan-A/MART-1 and gp100 peptides plus IFN-alpha results in the activation of specific CD8(+) T cells and monocyte/dendritic cell precursors. Cancer Res, 2006. 66(9): p. 4943-51.
156. Nakai, N., et al., Vaccination of Japanese patients with advanced melanoma with peptide, tumor lysate or both peptide and tumor lysate-pulsed mature, monocyte-derived dendritic cells. J Dermatol, 2006. 33(7): p. 462-72.
157. Palucka, A. K., et al., Dendritic cells loaded with killed allogeneic melanoma cells can induce objective clinical responses and MART-1 specific CD8+ T-cell immunity. J Immunother, 2006. 29(5): p. 545-57.
158. Lesimple, T., et al., Immunologic and clinical effects of injecting mature peptide-loaded dendritic cells by intralymphatic and intranodal routes in metastatic melanoma patients. Clin Cancer Res, 2006. 12(24): p. 7380-8.
159. Guo, J., et al., Intratumoral injection of dendritic cells in combination with local hyperthermia induces systemic antitumor effect in patients with advanced melanoma. Int J Cancer, 2007. 120(11): p. 2418-25.
160. O'Rourke, M. G., et al., Dendritic cell immunotherapy for stage IV melanoma. Melanoma Res, 2007. 17(5): p. 316-22.
161. Bercovici, N., et al., Analysis and characterization of antitumor T-cell response after administration of dendritic cells loaded with allogeneic tumor lysate to metastatic melanoma patients. J Immunother, 2008. 31(1): p. 101-12.
162. Hersey, P., et al., Phase I/II study of treatment with matured dendritic cells with or without low dose IL-2 in patients with disseminated melanoma. Cancer Immunol Immunother, 2008. 57(7): p. 1039-51.
163. von Euw, E. M., et al., A phase I clinical study of vaccination of melanoma patients with dendritic cells loaded with allogeneic apoptotic/necrotic melanoma cells. Analysis of toxicity and immune response to the vaccine and of IL-10-1082 promoter genotype as predictor of disease progression. J Transl Med, 2008. 6: p. 6.
164. Carrasco, J., et al., Vaccination of a melanoma patient with mature dendritic cells pulsed with MAGE-3 peptides triggers the activity of nonvaccine anti-tumor cells. J Immunol, 2008. 180(5): p. 3585-93.
165. Redman, B. G., et al., Phase 1b trial assessing autologous, tumor-pulsed dendritic cells as a vaccine administered with or without IL-2 in patients with metastatic melanoma. J Immunother, 2008. 31(6): p. 591-8.
166. Daud, A. I., et al., Phenotypic and functional analysis of dendritic cells and clinical outcome in patients with high-risk melanoma treated with adjuvant granulocyte macrophage colony-stimulating factor. J Clin Oncol, 2008. 26(19): p. 3235-41.
167. Engell-Noerregaard, L., et al., Review of clinical studies on dendritic cell-based vaccination of patients with malignant melanoma: assessment of correlation between clinical response and vaccine parameters. Cancer Immunol Immunother, 2009. 58(1): p. 1-14.
168. Nakai, N., et al., Immunohistological analysis of peptide-induced delayed-type hypersensitivity in advanced melanoma patients treated with melanoma antigen pulsed mature monocyte-derived dendritic cell vaccination. J Dermatol Sci, 2009. 53(1): p. 40-7.
169. Dillman, R. O., et al., Phase II trial of dendritic cells loaded with antigens from self-renewing, proliferating autologous tumor cells as patient-specific antitumor vaccines in patients with metastatic melanoma: final report. Cancer Biother Radiopharm, 2009. 24(3): p. 311-9.
170. Chang, J. W., et al., Immunotherapy with dendritic cells pulsed by autologous dactinomycin-induced melanoma apoptotic bodies for patients with malignant melanoma. Melanoma Res, 2009. 19(5): p. 309-15.
171. Trepiakas, R., et al., Vaccination with autologous dendritic cells pulsed with multiple tumor antigens for treatment of patients with malignant melanoma: results from a phase I/II trial. Cytotherapy, 2010. 12(6): p. 721-34.
172. Jacobs, J. F., et al., Dendritic cell vaccination in combination with anti-CD25 monoclonal antibody treatment: a phase I/II study in metastatic melanoma patients. Clin Cancer Res, 2010. 16(20): p. 5067-78.
173. Ribas, A., et al., Multicenter phase II study of matured dendritic cells pulsed with melanoma cell line lysates in patients with advanced melanoma. J Transl Med, 2010. 8: p. 89.
174. Ridolfi, L., et al., Unexpected high response rate to traditional therapy after dendritic cell-based vaccine in advanced melanoma: update of clinical outcome and subgroup analysis. Clin Dev Immunol, 2010. 2010: p. 504979.
175. Cornforth, A. N., et al., Resistance to the proapoptotic effects of interferon-gamma on melanoma cells used in patient-specific dendritic cell immunotherapy is associated with improved overall survival. Cancer Immunol Immunother, 2011. 60(1): p. 123-31.
176. Lesterhuis, W. J., et al., Wild-type and modified gp100 peptide-pulsed dendritic cell vaccination of advanced melanoma patients can lead to long-term clinical responses independent of the peptide used. Cancer Immunol Immunother, 2011. 60(2): p. 249-60.
177. Bjoern, J., et al., Changes in peripheral blood level of regulatory T cells in patients with malignant melanoma during treatment with dendritic cell vaccination and low-dose IL-2. Scand J Immunol, 2011. 73(3): p. 222-33.
178. Steele, J. C., et al., Phase I/II trial of a dendritic cell vaccine transfected with DNA encoding melan A and gp100 for patients with metastatic melanoma. Gene Ther, 2011. 18(6): p. 584-93.
179. Kim, D. S., et al., Immunotherapy of malignant melanoma with tumor lysate-pulsed autologous monocyte-derived dendritic cells. Yonsei Med J, 2011. 52(6): p. 990-8.
180. Ellebaek, E., et al., Metastatic melanoma patients treated with dendritic cell vaccination, Interleukin-2 and metronomic cyclophosphamide: results from a phase II trial. Cancer Immunol Immunother, 2012. 61(10): p. 1791-804.
181. Dillman, R. O., et al., Tumor stem cell antigens as consolidative active specific immunotherapy: a randomized phase II trial of dendritic cells versus tumor cells in patients with metastatic melanoma. J Immunother, 2012. 35(8): p. 641-9.
182. Dannull, J., et al., Melanoma immunotherapy using mature DCs expressing the constitutive proteasome. J Clin Invest, 2013. 123(7): p. 3135-45.
183. Finkelstein, S. E., et al., Combination of external beam radiotherapy (EBRT) with intratumoral injection of dendritic cells as neo-adjuvant treatment of high-risk soft tissue sarcoma patients. Int J Radiat Oncol Biol Phys, 2012. 82(2): p. 924-32.
184. Stift, A., et al., Dendritic cell vaccination in medullary thyroid carcinoma. Clin Cancer Res, 2004. 10(9): p. 2944-53.
185. Kuwabara, K., et al., Results of a phase I clinical study using dendritic cell vaccinations for thyroid cancer. Thyroid, 2007. 17(1): p. 53-8.
186. Bachleitner-Hofmann, T., et al., Pilot trial of autologous dendritic cells loaded with tumor lysate(s) from allogeneic tumor cell lines in patients with metastatic medullary thyroid carcinoma. Oncol Rep, 2009. 21(6): p. 1585-92.
187. Yu, J. S., et al., Vaccination of malignant glioma patients with peptide-pulsed dendritic cells elicits systemic cytotoxicity and intracranial T-cell infiltration. Cancer Res, 2001. 61(3): p. 842-7.
188. Yamanaka, R., et al., Vaccination of recurrent glioma patients with tumour lysate-pulsed dendritic cells elicits immune responses: results of a clinical phase I/II trial. Br J Cancer, 2003. 89(7): p. 1172-9.
189. Yu, J. S., et al., Vaccination with tumor lysate pulsed dendritic cells elicits antigen-specific, cytotoxic T-cells in patients with malignant glioma. Cancer Res, 2004. 64(14): p. 4973-9.
190. Yamanaka, R., et al., Tumor lysate and IL-18 loaded dendritic cells elicits Th1 response, tumor-specific CD8+ cytotoxic T cells in patients with malignant glioma. J Neurooncol, 2005. 72(2): p. 107-13.
191. Yamanaka, R., et al., Clinical evaluation of dendritic cell vaccination for patients with recurrent glioma: results of a clinical phase I/II trial. Clin Cancer Res, 2005. 11(11): p. 4160-7.
192. Liau, L. M., et al., Dendritic cell vaccination in glioblastoma patients induces systemic and intracranial T-cell responses modulated by the local central nervous system tumor microenvironment. Clin Cancer Res, 2005. 11(15): p. 5515-25.
193. Walker, D. G., et al., Results of a phase I dendritic cell vaccine trial for malignant astrocytoma: potential interaction with adjuvant chemotherapy. J Clin Neurosci, 2008. 15(2): p. 114-21.
194. Leplina, O. Y., et al., Use of interferon-alpha-induced dendritic cells in the therapy of patients with malignant brain gliomas. Bull Exp Biol Med, 2007. 143(4): p. 528-34.
195. De Vleeschouwer, S., et al., Postoperative adjuvant dendritic cell-based immunotherapy in patients with relapsed glioblastoma multiforme. Clin Cancer Res, 2008. 14(10): p. 3098-104.
196. Ardon, H., et al., Adjuvant dendritic cell-based tumour vaccination for children with malignant brain tumours. Pediatr Blood Cancer, 2010. 54(4): p. 519-25.
197. Prins, R. M., et al., Gene expression profile correlates with T-cell infiltration and relative survival in glioblastoma patients vaccinated with dendritic cell immunotherapy. Clin Cancer Res, 2011. 17(6): p. 1603-15.
198. Okada, H., et al., Induction of CD8+ T-cell responses against novel glioma-associated antigen peptides and clinical activity by vaccinations with {alpha}-type 1 polarized dendritic cells and polyinosinic-polycytidylic acid stabilized by lysine and carboxymethylcellulose in patients with recurrent malignant glioma. J Clin Oncol, 2011. 29(3): p. 330-6.
199. Fadul, C. E., et al., Immune response in patients with newly diagnosed glioblastoma multiforme treated with intranodal autologous tumor lysate-dendritic cell vaccination after radiation chemotherapy. J Immunother, 2011. 34(4): p. 382-9.
200. Chang, C. N., et al., A phase I/II clinical trial investigating the adverse and therapeutic effects of a postoperative autologous dendritic cell tumor vaccine in patients with malignant glioma. J Clin Neurosci, 2011. 18(8): p. 1048-54.
201. Cho, D. Y., et al., Adjuvant immunotherapy with whole-cell lysate dendritic cells vaccine for glioblastoma multiforme: a phase II clinical trial. World Neurosurg, 2012. 77(5-6): p. 736-44.
202. Iwami, K., et al., Peptide-pulsed dendritic cell vaccination targeting interleukin-13 receptor alpha2 chain in recurrent malignant glioma patients with HLA-A*24/A*02 allele. Cytotherapy, 2012. 14(6): p. 733-42.
203. Fong, B., et al., Monitoring of regulatory T cell frequencies and expression of CTLA-4 on T cells, before and after DC vaccination, can predict survival in GBM patients. PLoS One, 2012. 7(4): p. e32614.
204. De Vleeschouwer, S., et al., Stratification according to HGG-IMMUNO RPA model predicts outcome in a large group of patients with relapsed malignant glioma treated by adjuvant postoperative dendritic cell vaccination. Cancer Immunol Immunother, 2012. 61(11): p. 2105-12.
205. Phuphanich, S., et al., Phase I trial of a multi-epitope-pulsed dendritic cell vaccine for patients with newly diagnosed glioblastoma. Cancer Immunol Immunother, 2013. 62(1): p. 125-35.
206. Akiyama, Y., et al., alpha-type-1 polarized dendritic cell-based vaccination in recurrent high-grade glioma: a phase I clinical trial. BMC Cancer, 2012. 12: p. 623.
207. Prins, R. M., et al., Comparison of glioma-associated antigen peptide-loaded versus autologous tumor lysate-loaded dendritic cell vaccination in malignant glioma patients. J Immunother, 2013. 36(2): p. 152-7.
208. Shah, A. H., et al., Dendritic cell vaccine for recurrent high-grade gliomas in pediatric and adult subjects: clinical trial protocol. Neurosurgery, 2013. 73(5): p. 863-7.
209. Reichardt, V. L., et al., Idiotype vaccination using dendritic cells after autologous peripheral blood stem cell transplantation for multiple myeloma—a feasibility study. Blood, 1999. 93(7): p. 2411-9.
210. Lim, S. H. and R. Bailey-Wood, Idiotypic protein pulsed dendritic cell vaccination in multiple myeloma. Int J Cancer, 1999. 83(2): p. 215-22.
211. Motta, M. R., et al., Generation of dendritic cells from CD14+ monocytes positively selected by immunomagnetic adsorption for multiple myeloma patients enrolled in a clinical trial of anti-idiotype vaccination. Br J Haematol, 2003. 121(2): p. 240-50.
212. Reichardt, V. L., et al., Idiotype vaccination of multiple myeloma patients using monocyte-derived dendritic cells. Haematologica, 2003. 88(10): p. 1139-49.
213. Guardino, A. E., et al., Production of myeloid dendritic cells (DC) pulsed with tumor-specific idiotype protein for vaccination of patients with multiple myeloma. Cytotherapy, 2006. 8(3): p. 277-89.
214. Lacy, M. Q., et al., Idiotype-pulsed antigen presenting cells following autologous transplantation for multiple myeloma may be associated with prolonged survival. Am J Hematol, 2009. 84(12): p. 799-802.
215. Yi, Q., et al., Optimizing dendritic cell-based immunotherapy in multiple myeloma: intranodal injections of idiotype pulsed CD40 ligand-matured vaccines led to induction of type-1 and cytotoxic T-cell immune responses in patients. Br J Haematol, 2010. 150(5): p. 554-64.
216. Rollig, C., et al., Induction of cellular immune responses in patients with stage-I multiple myeloma after vaccination with autologous idiotype pulsed dendritic cells. J Immunother, 2011. 34(1): p. 100-6.
217. Zahradova, L., et al., Efficacy and safety of Id-protein-loaded dendritic cell vaccine in patients with multiple myeloma—phase II study results. Neoplasma, 2012. 59(4): p. 440-9.
218. Timmerman, J. M., et al., Idiotype pulsed dendritic cell vaccination for B-cell lymphoma: clinical and immune responses in 35 patients. Blood, 2002. 99(5): p. 1517-26.
219. Maier, T., et al., Vaccination of patients with cutaneous T-cell lymphoma using intranodal injection of autologous tumor-lysate-pulsed dendritic cells. Blood, 2003. 102(7): p. 2338-44.
220. Di Nicola, M., et al., Vaccination with autologous tumor-loaded dendritic cells induces clinical and immunologic responses in indolent B-cell lymphoma patients with relapsed and measurable disease: a pilot study. Blood, 2009. 113(1): p. 18-27.
221. Hus, I., et al., Allogeneic dendritic cells pulsed with tumor lysates or apoptotic bodies as immunotherapy for patients with early-stage B-cell chronic lymphocytic leukemia. Leukemia, 2005. 19(9): p. 1621-7.
222. Li, L., et al., Immunotherapy for patients with acute myeloid leukemia using autologous dendritic cells generated from leukemic blasts. Int J Oncol, 2006. 28(4): p. 855-61.
223. Roddie, H., et al., Phase I/II study of vaccination with dendritic-like leukaemia cells for the immunotherapy of acute myeloid leukaemia. Br J Haematol, 2006. 133(2): p. 152-7.
224. Litzow, M. R., et al., Testing the safety of clinical-grade mature autologous myeloid DC in a phase I clinical immunotherapy trial of CML. Cytotherapy, 2006. 8(3): p. 290-8.
225. Westermann, J., et al., Vaccination with autologous non-irradiated dendritic cells in patients with bcr/abl+ chronic myeloid leukaemia. Br J Haematol, 2007. 137(4): p. 297-306.
226. Hus, I., et al., Vaccination of B-CLL patients with autologous dendritic cells can change the frequency of leukemia antigen-specific CD8+ T cells as well as CD4+ CD25+ FoxP3+ regulatory T cells toward an antileukemia response. Leukemia, 2008. 22(5): p. 1007-17.
227. Palma, M., et al., Development of a dendritic cell-based vaccine for chronic lymphocytic leukemia. Cancer Immunol Immunother, 2008. 57(11): p. 1705-10.
228. Van Tendeloo, V. F., et al., Induction of complete and molecular remissions in acute myeloid leukemia by Wilms' tumor 1 antigen-targeted dendritic cell vaccination. Proc Natl Acad Sci USA, 2010. 107(31): p. 13824-9.
229. Iwashita, Y., et al., A phase I study of autologous dendritic cell-based immunotherapy for patients with unresectable primary liver cancer. Cancer Immunol Immunother, 2003. 52(3): p. 155-61.
230. Lee, W. C., et al., Vaccination of advanced hepatocellular carcinoma patients with tumor lysate pulsed dendritic cells: a clinical trial. J Immunother, 2005. 28(5): p. 496-504.
231. Butterfield, L. H., et al., A phase I/II trial testing immunization of hepatocellular carcinoma patients with dendritic cells pulsed with four alpha-fetoprotein peptides. Clin Cancer Res, 2006. 12(9): p. 2817-25.
232. Palmer, D. H., et al., A phase II study of adoptive immunotherapy using dendritic cells pulsed with tumor lysate in patients with hepatocellular carcinoma. Hepatology, 2009. 49(1): p. 124-32.
233. El Ansary, M., et al., Immunotherapy by autologous dendritic cell vaccine in patients with advanced HCC. J Cancer Res Clin Oncol, 2013. 139(1): p. 39-48.
234. Tada, F., et al., Phase I/II study of immunotherapy using tumor antigen-pulsed dendritic cells in patients with hepatocellular carcinoma. Int J Oncol, 2012. 41(5): p. 1601-9.
235. Ueda, Y., et al., Dendritic cell-based immunotherapy of cancer with carcinoembryonic antigen-derived, HLA-A24-restricted CTL epitope: Clinical outcomes of 18 patients with metastatic gastrointestinal or lung adenocarcinomas. Int J Oncol, 2004. 24(4): p. 909-17.
236. Hirschowitz, E. A., et al., Autologous dendritic cell vaccines for non-small-cell lung cancer. J Clin Oncol, 2004. 22(14): p. 2808-15.
237. Chang, G. C., et al., A pilot clinical trial of vaccination with dendritic cells pulsed with autologous tumor cells derived from malignant pleural effusion in patients with late-stage lung carcinoma. Cancer, 2005. 103(4): p. 763-71.
238. Yannelli, J. R., et al., The large scale generation of dendritic cells for the immunization of patients with non-small cell lung cancer (NSCLC). Lung Cancer, 2005. 47(3): p. 337-50.
239. Ishikawa, A., et al., A phase I study of alpha-galactosylceramide (KRN7000)-pulsed dendritic cells in patients with advanced and recurrent non-small cell lung cancer. Clin Cancer Res, 2005. 11(5): p. 1910-7.
240. Antonia, S. J., et al., Combination of p53 cancer vaccine with chemotherapy in patients with extensive stage small cell lung cancer. Clin Cancer Res, 2006. 12(3 Pt 1): p. 878-87.
241. Perrot, I., et al., Dendritic cells infiltrating human non-small cell lung cancer are blocked at immature stage. J Immunol, 2007. 178(5): p. 2763-9.
242. Hirschowitz, E. A., et al., Immunization of NSCLC patients with antigen-pulsed immature autologous dendritic cells. Lung Cancer, 2007. 57(3): p. 365-72.
243. Baratelli, F., et al., Pre-clinical characterization of GMP grade CCL21-gene modified dendritic cells for application in a phase I trial in non-small cell lung cancer. J Transl Med, 2008. 6: p. 38.
244. Hegmans, J. P., et al., Consolidative dendritic cell-based immunotherapy elicits cytotoxicity against malignant mesothelioma. Am J Respir Crit Care Med, 2010. 181(12): p. 1383-90.
245. Um, S. J., et al., Phase I study of autologous dendritic cell tumor vaccine in patients with non-small cell lung cancer. Lung Cancer, 2010. 70(2): p. 188-94.
246. Chiappori, A. A., et al., INGN-225: a dendritic cell-based p53 vaccine (Ad.p53-DC) in small cell lung cancer: observed association between immune response and enhanced chemotherapy effect. Expert Opin Biol Ther, 2010. 10(6): p. 983-91.
247. Perroud, M. W., Jr., et al., Mature autologous dendritic cell vaccines in advanced non-small cell lung cancer: a phase I pilot study. J Exp Clin Cancer Res, 2011. 30: p. 65.
248. Skachkova, O. V., et al., Immunological markers of anti-tumor dendritic cells vaccine efficiency in patients with non-small cell lung cancer. Exp Oncol, 2013. 35(2): p. 109-13.
249. Hernando, J. J., et al., Vaccination with autologous tumour antigen pulsed dendritic cells in advanced gynaecological malignancies: clinical and immunological evaluation of a phase I trial. Cancer Immunol Immunother, 2002. 51(1): p. 45-52.
250. Rahma, O. E., et al., A gynecologic oncology group phase II trial of two p53 peptide vaccine approaches: subcutaneous injection and intravenous pulsed dendritic cells in high recurrence risk ovarian cancer patients. Cancer Immunol Immunother, 2012. 61(3): p. 373-84.
251. Chu, C. S., et al., Phase I/II randomized trial of dendritic cell vaccination with or without cyclophosphamide for consolidation therapy of advanced ovarian cancer in first or second remission. Cancer Immunol Immunother, 2012. 61(5): p. 629-41.
252. Kandalaft, L. E., et al., A Phase I vaccine trial using dendritic cells pulsed with autologous oxidized lysate for recurrent ovarian cancer. J Transl Med, 2013. 11: p. 149.
253. Lepisto, A. J., et al., A phase I/II study of a MUC1 peptide pulsed autologous dendritic cell vaccine as adjuvant therapy in patients with resected pancreatic and biliary tumors. Cancer Ther, 2008. 6(B): p. 955-964.
254. Rong, Y., et al., A phase I pilot trial of MUC1-peptide-pulsed dendritic cells in the treatment of advanced pancreatic cancer. Clin Exp Med, 2012. 12(3): p. 173-80.
255. Endo, H., et al., Phase I trial of preoperative intratumoral injection of immature dendritic cells and OK-432 for resectable pancreatic cancer patients. J Hepatobiliary Pancreat Sci, 2012. 19(4): p. 465-75.
256. Zumwalt, T. J., et al., Active secretion of CXCL10 and CCL5 from colorectal cancer microenvironments associates with GranzymeB+ CD8+ T-cell infiltration. Oncotarget, 2015. 6(5): p. 2981-91.
257. Ochsenbein, A. F., Principles of tumor immunosurveillance and implications for immunotherapy. Cancer Gene Ther, 2002. 9(12): p. 1043-55.
258. Ochsenbein, A. F., et al., Roles of tumour localization, second signals and cross priming in cytotoxic T-cell induction. Nature, 2001. 411(6841): p. 1058-64.
259. Buhtoiarov, I. N., et al., CD40 ligation activates murine macrophages via an IFN-gamma-dependent mechanism resulting in tumor cell destruction in vitro. J Immunol, 2005. 174(10): p. 6013-22.
260. Egilmez, N. K., et al., Human CD4+ effector T cells mediate indirect interleukin-12- and interferon-gamma-dependent suppression of autologous HLA-negative lung tumor xenografts in severe combined immunodeficient mice. Cancer Res, 2002. 62(9): p. 2611-7.
261. Pace, J. L., et al., Recombinant mouse gamma interferon induces the priming step in macrophage activation for tumor cell killing. J Immunol, 1983. 130(5): p. 2011-3.
262. Heusinkveld, M., et al., M2 macrophages induced by prostaglandin E2 and IL-6 from cervical carcinoma are switched to activated M1 macrophages by CD4+ Th1 cells. J Immunol, 2011. 187(3): p. 1157-65.
263. Hu, G. and S. Wang, Tumor-infiltrating CD45RO+ Memory T Lymphocytes Predict Favorable Clinical Outcome in Solid Tumors. Sci Rep, 2017. 7(1): p. 10376.
264. Lohneis, P., et al., Cytotoxic tumour-infiltrating T lymphocytes influence outcome in resected pancreatic ductal adenocarcinoma. Eur J Cancer, 2017. 83: p. 290-301.
265. Liu, S., et al., Role of Cytotoxic Tumor-Infiltrating Lymphocytes in Predicting Outcomes in Metastatic HER2-Positive Breast Cancer: A Secondary Analysis of a Randomized Clinical Trial. JAMA Oncol, 2017: p. e172085.
266. Berntsson, J., et al., The clinical impact of tumour-infiltrating lymphocytes in colorectal cancer differs by anatomical subsite: A cohort study. Int J Cancer, 2017. 141(8): p. 1654-1666.
267. Xu, Y., et al., Higher Numbers of T-Bet+ Tumor-Infiltrating Lymphocytes Associate with Better Survival in Human Epithelial Ovarian Cancer. Cell Physiol Biochem, 2017. 41(2): p. 475-483.
268. Melief, S. M., et al., Long-term Survival and Clinical Benefit from Adoptive T-cell Transfer in Stage IV Melanoma Patients Is Determined by a Four-Parameter Tumor Immune Signature. Cancer Immunol Res, 2017. 5(2): p. 170-179.
269. Scurr, M. J., et al., Low-dose cyclophosphamide induces anti-tumor T-cell responses which associate with survival in metastatic colorectal cancer. Clin Cancer Res, 2017.
270. Wang, L., et al., Arsenic trioxide is an immune adjuvant in liver cancer treatment. Mol Immunol, 2017. 81: p. 118-126.
271. Ouyang, Z., et al., Regulatory T cells in the immunotherapy of melanoma. Tumour Biol, 2016. 37(1): p. 77-85.
272. Dimeloe, S., et al., Human regulatory T cells lack the cyclophosphamide-extruding transporter ABCB1 and are more susceptible to cyclophosphamide-induced apoptosis. Eur J Immunol, 2014. 44(12): p. 3614-20.
273. Camisaschi, C., et al., Effects of cyclophosphamide and IL-2 on regulatory CD4+ T cell frequency and function in melanoma patients vaccinated with HLA-class I peptides: impact on the antigen-specific T cell response. Cancer Immunol Immunother, 2013. 62(5): p. 897-908.
274. Kan, S., et al., Suppressive effects of cyclophosphamide and gemcitabine on regulatory T-cell induction in vitro. Anticancer Res, 2012. 32(12): p. 5363-9.
275. Farsam, V., et al., Antitumor and immunomodulatory properties of artemether and its ability to reduce CD4+ CD25+ FoxP3+ T reg cells in vivo. Int Immunopharmacol, 2011. 11(11): p. 1802-8.
276. Lawrence, H. S. and A. M. Pappenheimer, Jr., Transfer of delayed hypersensitivity to diphtheria toxin in man. J Exp Med, 1956. 104(3): p. 321-35.
277. Rosenfeld, S. and D. Dressler, Transfer factor: a subcellular component that transmits information for specific immune responses. Proc Natl Acad Sci USA, 1974. 71(6): p. 2473-7.
278. Dressler, D. and S. Rosenfeld, On the chemical nature of transfer factor. Proc Natl Acad Sci USA, 1974. 71(11): p. 4429-34.
279. Shifrine, M. and R. Scibienski, Transfer factor—hypotheses for its structure and function. Oncology, 1975. 32(5-6): p. 269-74.
280. Kirkpatrick, C. H., Properties and activities of transfer factor. J Allergy Clin Immunol, 1975. 55(6): p. 411-21.
281. Burger, D. R., et al., Human transfer factor fractionation and biologic activity. J Immunol, 1976. 117(3): p. 789-96.
282. Berron-Perez, R., et al., Indications, usage, and dosage of the transfer factor. Rev Alerg Mex, 2007. 54(4): p. 134-9.
283. Alexandrescu, D. T., C. A. Dasanu, and C. L. Kauffman, Acute scurvy during treatment with interleukin-2. Clin Exp Dermatol, 2009. 34(7): p. 811-4.
284. Anthony, H. M. and C. J. Schorah, Severe hypovitaminosis C in lung-cancer patients: the utilization of vitamin C in surgical repair and lymphocyte-related host resistance. Br J Cancer, 1982. 46(3): p. 354-67.
285. McMurray, D. N., Cell-mediated immunity in nutritional deficiency. Prog Food Nutr Sci, 1984. 8(3-4): p. 193-228.
286. http//www.highbeam.com/doc/1G1-186526887.html.
287. Marcus, S. L., et al., Severe hypovitaminosis C occurring as the result of adoptive immunotherapy with high-dose interleukin 2 and lymphokine-activated killer cells. Cancer Res, 1987. 47(15): p. 4208-12.
288. Marcus, S. L., et al., Hypovitaminosis C in patients treated with high-dose interleukin 2 and lymphokine-activated killer cells. Am J Clin Nutr, 1991. 54(6 Suppl): p. 1292S-1297S.
289. Yeom, C. H., G. C. Jung, and K. J. Song, Changes of terminal cancer patients' health-related quality of life after high dose vitamin C administration. J Korean Med Sci, 2007. 22(1): p. 7-11.
290. Murata, A., F. Morishige, and H. Yamaguchi, Prolongation of survival times of terminal cancer patients by administration of large doses of ascorbate. Int J Vitam Nutr Res Suppl, 1982. 23: p. 103-13.
291. Cameron, E. and A. Campbell, The orthomolecular treatment of cancer. II. Clinical trial of high-dose ascorbic acid supplements in advanced human cancer. Chem Biol Interact, 1974. 9(4): p. 285-315.
292. Riordan, N. H., et al., Intravenous ascorbate as a tumor cytotoxic chemotherapeutic agent. Med Hypotheses, 1995. 44(3): p. 207-13.
293. Deubzer, B., et al., H(2)O(2)-mediated cytotoxicity of pharmacologic ascorbate concentrations to neuroblastoma cells: potential role of lactate and ferritin. Cell Physiol Biochem. 25(6): p. 767-74.
294. Gilloteaux, J., et al., Cell damage and death by autoschizis in human bladder (RT4) carcinoma cells resulting from treatment with ascorbate and menadione. Ultrastruct Pathol. 34(3): p. 140-60.
295. Cullen, J. J., Ascorbate induces autophagy in pancreatic cancer. Autophagy. 6(3): p. 421-2.
296. Takemura, Y., et al., High dose of ascorbic acid induces cell death in mesothelioma cells. Biochem Biophys Res Commun. 394(2): p. 249-53.
297. Verrax, J., et al., In situ modulation of oxidative stress: a novel and efficient strategy to kill cancer cells. Curr Med Chem, 2009. 16(15): p. 1821-30.
298. Fromberg, A., et al., Ascorbate exerts anti-proliferative effects through cell cycle inhibition and sensitizes tumor cells towards cytostatic drugs. Cancer Chemother Pharmacol.
299. Pollard, H. B., et al., Pharmacological ascorbic acid suppresses syngeneic tumor growth and metastases in hormone-refractory prostate cancer. In vivo. 24(3): p. 249-55.
300. Chen, Q., et al., Pharmacologic doses of ascorbate act as a prooxidant and decrease growth of aggressive tumor xenografts in mice. Proc Natl Acad Sci USA, 2008. 105(32): p. 11105-9.
301. Padayatty, S. J., et al., Intravenously administered vitamin Cas cancer therapy: three cases. CMAJ, 2006. 174(7): p. 937-42.
302. Mikirova, N. A., T. E. Ichim, and N. H. Riordan, Anti-angiogenic effect of high doses of ascorbic acid. J Transl Med, 2008. 6: p. 50.
303. Ashino, H., et al., Novel function of ascorbic acid as an angiostatic factor. Angiogenesis, 2003. 6(4): p. 259-69.
304. Mikirova, N. A., J. J. Casciari, and N. H. Riordan, Ascorbate inhibition of angiogenesis in aortic rings ex vivo and subcutaneous Matrigel plugs in vivo. J Angiogenes Res. 2: p. 2.
305. Yeom, C. H., et al., High dose concentration administration of ascorbic acid inhibits tumor growth in BALB/C mice implanted with sarcoma 180 cancer cells via the restriction of angiogenesis. J Transl Med, 2009. 7: p. 70.
306. Muellner, M. K., et al., Vitamin C inhibits NO-induced stabilization of HIF-1alpha in HUVECs. Free Radic Res. 44(7): p. 783-91.
307. Horak, P., et al., Negative feedback control of HIF-1 through REDD1-regulated ROS suppresses tumorigenesis. Proc Natl Acad Sci USA. 107(10): p. 4675-80.
308. Gao, P., et al., HIF-dependent antitumorigenic effect of antioxidants in vivo. Cancer Cell, 2007. 12(3): p. 230-8.
309. Kuiper, C., et al., Low ascorbate levels are associated with increased hypoxia-inducible factor-1 activity and an aggressive tumor phenotype in endometrial cancer. Cancer Res. 70(14): p. 5749-58.
310. http://www.clinicaltrials.gov/ct2/show/NCT00441207.
311. http://www.clinicaltrials.gov/ct2/show/NCT01080352.
312. http://www.clinicaltrials.gov/ct2/show/NCT00626444.
313. http://www.clinicaltrials.gov/ct2/show/NCT01125449.
314. http://www.clinicaltrials.gov/ct2/show/NCT01050621.
315. http://www.clinicaltrials.gov/ct2/show/NCT00954525.
316. Tatla, S., et al., The role of reactive oxygen species in triggering proliferation and IL-2 secretion in T cells. Free Radic Biol Med, 1999. 26(1-2): p. 14-24.
317. Williams, M. S. and J. Kwon, T cell receptor stimulation, reactive oxygen species, and cell signaling. Free Radic Biol Med, 2004. 37(8): p. 1144-51.
318. Schwager, J. and J. Schulze, Influence of ascorbic acid on the response to mitogens and interleukin production of porcine lymphocytes. Int J Vitam Nutr Res, 1997. 67(1): p. 10-6.
319. Eylar, E., et al., Sustained levels of ascorbic acid are toxic and immunosuppressive for human T cells. P R Health Sci J, 1996. 15(1): p. 21-6.
320. Huwyler, T., A. Hirt, and A. Morell, Effect of ascorbic acid on human natural killer cells. Immunol Lett, 1985. 10(3-4): p. 173-6.
321. Tan, P. H., et al., Inhibition of NF-kappa B and oxidative pathways in human dendritic cells by antioxidative vitamins generates regulatory T cells. J Immunol, 2005. 174(12): p. 7633-44.
322. Chen, S., L. Yang, and Y. Li, TCR zeta chain expression in T cells from patients with CML. Hematology, 2009. 14(2): p. 95-100.
323. Kulkarni, D. P., et al., Mechanisms involved in the down-regulation of TCR zeta chain in tumor versus peripheral blood of oral cancer patients. Int J Cancer, 2009. 124(7): p. 1605-13.
324. Gruber, I. V., et al., Down-regulation of CD28, TCR-zeta (zeta) and up-regulation of FAS in peripheral cytotoxic T-cells of primary breast cancer patients. Anticancer Res, 2008. 28(2A): p. 779-84.
325. Pignataro, L., et al., Down-regulation of zeta chain and zeta-associated protein 70 (Zap 70) expression in circulating T lymphocytes in laryngeal squamous cell carcinoma. Anal Quant Cytol Histol, 2007. 29(1): p. 57-62.
326. Zehbe, I., et al., Different T-cell receptor (TCR) zeta chain expression in cervical cancer and its precursor lesions. Zentralbl Gynakol, 2006. 128(5): p. 266-70.
327. Ciszak, L., et al., Alterations in the expression of signal-transducing CD3 zeta chain in T cells from patients with chronic inflammatory/autoimmune diseases. Arch Immunol Ther Exp (Warsz), 2007. 55(6): p. 373-86.
328. Baniyash, M., TCR zeta-chain downregulation: curtailing an excessive inflammatory immune response. Nat Rev Immunol, 2004. 4(9): p. 675-87.
329. Pitcher, L. A. and N. S. van Oers, T-cell receptor signal transmission: who gives an ITAM? Trends Immunol, 2003. 24(10): p. 554-60.
330. Gastman, B. R., et al., Tumor-induced apoptosis of T lymphocytes: elucidation of intracellular apoptotic events. Blood, 2000. 95(6): p. 2015-23.
331. Boussiotis, V. A., et al., Differential association of protein tyrosine kinases with the T cell receptor is linked to the induction of anergy and its prevention by B7 family-mediated costimulation. J Exp Med, 1996. 184(2): p. 365-76.
332. Kim, C. W., et al., Alteration of signal-transducing molecules and phenotypical characteristics in peripheral blood lymphocytes from gastric carcinoma patients. Pathobiology, 1999. 67(3): p. 123-8.
333. Reichert, T. E., et al., Absent or low expression of the zeta chain in T cells at the tumor site correlates with poor survival in patients with oral carcinoma. Cancer Res, 1998. 58(23): p. 5344-7.
334. Zea, A. H., et al., Alterations in T cell receptor and signal transduction molecules in melanoma patients. Clin Cancer Res, 1995. 1(11): p. 1327-35.
335. Healy, C. G., et al., Impaired expression and function of signal-transducing zeta chains in peripheral T cells and natural killer cells in patients with prostate cancer. Cytometry, 1998. 32(2): p. 109-19.
336. Mulder, W. M., et al., T cell receptor-zeta and granzyme B expression in mononuclear cell infiltrates in normal colon mucosa and colon carcinoma. Gut, 1997. 40(1): p. 113-9.
337. Muller, D., et al., [The expression of zeta-chain of the T cell receptor as prognostic marker for patients with head and neck cancer]. Laryngorhinootologie, 2002. 81(7): p. 516-20.
338. Whiteside, T. L., Down-regulation of zeta-chain expression in T cells: a biomarker of prognosis in cancer? Cancer Immunol Immunother, 2004. 53(10): p. 865-78.
339. Eleftheriadis, T., et al., Decreased CD3+ CD16+ natural killer-like T-cell percentage and zeta-chain expression accompany chronic inflammation in haemodialysis patients. Nephrology (Carlton), 2009. 14(5): p. 471-5.
340. Eleftheriadis, T., et al., Chronic inflammation and CD16+ natural killer cell zeta-chain downregulation in hemodialysis patients. Blood Purif, 2008. 26(4): p. 317-21.
341. Nambiar, M. P., et al., TCR zeta-chain abnormalities in human systemic lupus erythematosus. Methods Mol Med, 2004. 102: p. 49-72.
342. Takeuchi, T., et al., T cell abnormalities in systemic lupus erythematosus. Autoimmunity, 2005. 38(5): p. 339-46.
343. Berg, L., et al., Down-regulation of the T cell receptor CD3 zeta chain in rheumatoid arthritis (RA) and its influence on T cell responsiveness. Clin Exp Immunol, 2000. 120(1): p. 174-82.
344. Maurice, M. M., et al., Defective TCR-mediated signaling in synovial T cells in rheumatoid arthritis. J Immunol, 1997. 159(6): p. 2973-8.
345. Ammirati, E., et al., Expansion of T-cell receptor zeta dim effector T cells in acute coronary syndromes. Arterioscler Thromb Vasc Biol, 2008. 28(12): p. 2305-11.
346. Sikora, J., et al., The role of monocytes/macrophages in TCR-zeta chain downregulation and apoptosis of T lymphocytes in malignant pleural effusions. J Biol Regul Homeost Agents, 2004. 18(1): p. 26-32.
347. Markiewski, M. M., et al., Modulation of the antitumor immune response by complement. Nat Immunol, 2008. 9(11): p. 1225-35.
348. Corzo, C. A., et al., Mechanism regulating reactive oxygen species in tumor-induced myeloid-derived suppressor cells. J Immunol, 2009. 182(9): p. 5693-701.
349. Choi, J. Y., J. A. Oughton, and N. I. Kerkvliet, Functional alterations in CD11b(+)Gr-1(+) cells in mice injected with allogeneic tumor cells and treated with 2,3,7,8-tetrachlorodibenzo-p-dioxin. Int Immunopharmacol, 2003. 3(4): p. 553-70.
350. Makarenkova, V. P., et al., CD11b+/Gr-1+ myeloid suppressor cells cause T cell dysfunction after traumatic stress. J Immunol, 2006. 176(4): p. 2085-94.
351. Ezernitchi, A. V., et al., TCR zeta down-regulation under chronic inflammation is mediated by myeloid suppressor cells differentially distributed between various lymphatic organs. J Immunol, 2006. 177(7): p. 4763-72.
352. Schmielau, J. and O. J. Finn, Activated granulocytes and granulocyte-derived hydrogen peroxide are the underlying mechanism of suppression of t-cell function in advanced cancer patients. Cancer Res, 2001. 61(12): p. 4756-60.
353. Nambiar, M. P., et al., Oxidative stress is involved in the heat stress-induced downregulation of TCR zeta chain expression and TCR/CD3-mediated [Ca(2+)](i) response in human T-lymphocytes. Cell Immunol, 2002. 215(2): p. 151-61.

What is claimed is:

1. A method of treating an individual for cancer, comprising the steps of:
a) administering one or more immunogenic compositions to the individual;
b) administering a therapeutically effective amount of fibroblast cells to the individual;
c) optionally administering one or more compositions capable of augmenting antigen presentation in cells of the cancer; and
d) optionally administering one or more compositions and/or actions capable of inducing cancer cell death.

2. The method of claim 1, wherein steps a)-d) are each performed concurrently, independently, or in any combination thereof;

3. The method of claim 1 or 2, wherein steps a)-d) are performed in any order;

4. The method of any one of claims 1-3, wherein step a) is performed once or repeatedly; wherein step b) is performed once or repeatedly; wherein step c) is performed once or repeatedly; wherein step d) is performed once or repeatedly.

5. The method of any one of claims 1-4, wherein step a) is performed at least once prior to step b).

6. The method of claim 5, wherein step a) is also performed at least once after step b).

7. The method of any one of claims 1-4, wherein step b) is performed at least once prior to step c).

8. The method of claim 7, wherein step b) is also performed at least once after step a).

9. The method of any one of claims 1-8, wherein steps c) and/or d) are performed at least once.

10. The method of any one of claims 1-8, wherein steps c) and/or d) are not performed.

11. The method of any one of claims 1-10, wherein step a) is performed at least once followed by performing step b) at least once followed by performing step a) at least once.

12. The method of claim 11, wherein steps c) and/or d) are performed at any point in relation to steps a) and b).

13. The method of any one of claims 1-10, wherein step b) is performed at least once followed by performing step a) at least once followed by performing step b) at least once.

14. The method of claim 13, wherein steps c) and/or d) are performed at any point in relation to steps a) and b).

15. The method of any one of claims 1-14, wherein the fibroblasts reduce tumor-associated immune suppression.

16. The method of any one of claims 1-15, wherein the fibroblasts possess anti-inflammatory activity.

17. The method of claim 16, wherein anti-inflammatory activity comprises suppressing TNF-alpha production from the tumor microenvironment, suppressing production of interleukin (IL)-1, suppressing production of IL-6, or a combination thereof.

18. The method of any one of claims 1-17, wherein the fibroblasts are cultured under conditions promoting the ability of said fibroblasts to reduce production of one or more inflammatory mediators.

19. The method of claim 18, wherein said culture comprises exposure to tissue culture additives selected from the group consisting of IL-10, indomethacin, valproic acid, low dose naltrexone, interleukin-27, and a combination thereof.

20. The method of claim 18, wherein the inflammatory mediator is selected from the group consisting of TNF-alpha, TNF-beta, IL-33, interferon gamma, interferon beta, HMGB1, and a combination thereof.

21. The method of any one of claims 1-20, wherein the fibroblasts express at least one marker selected from the group consisting of CD117, CD105, Oct-4, CD-34, KLF-4, Nanog, Sox-2, Rex-1, GDF-3, Stella, GDF-11, and a combination thereof.

22. The method of any one of claims 1-21, wherein the fibroblasts comprise the expression of flu peptides.

23. The method of any one of claims 1-22, wherein the fibroblasts are dedifferentiated.

24. The method of claim 23, wherein the fibroblasts are re-differentiated following dedifferentiation.

25. The method of any one of claims 1-24, wherein the fibroblasts express one or more antigens present on at least one cancer cell in the individual.

26. The method of claim 25, wherein the antigen expressed by the fibroblasts is selected from the group consisting of Fos-related antigen 1, LCK, FAP, VEGFR2, NA17, PDGFR-beta, PAP, MAD-CT-2, Tie-2, PSA, protamine 2, legumain, endosialin, prostate stem cell antigen, carbonic anhydrase IX, STn, Page4, proteinase 3, GM3 ganglioside, tyrosinase, MART1, gp100, SART3, RGS5, SSX2, Globol1, Tn, CEA, hCG, PRAME, XAGE-1, AKAP-4, TRP-2, B7H3, sperm fibrous sheath protein, CYP1B1, HMWMAA, sLe(a), MAGE A1, GD2, PSMA, mesothelin, fucosyl GM1, GD3, sperm protein 17, NY-ESO-1, PAX5, AFP, polysialic acid, EpCAM, MAGE-A3, mutant p53, ras, mutant ras, NY-BR1, PAX3, HER2/neu, OY-TES1, HPV E6 E7, PLAC1, hTERT, BORIS, ML-IAP, idiotype of b cell lymphoma or multiple myeloma, EphA2, EGFRvIII, cyclin B1, RhoC, androgen receptor, surviving, MYCN, wildtype p53, LMP2, ETV6-AML, MUC1, BCR-ABL, ALK, WT1, ERG (TMPRSS2 ETS fusion gene), sarcoma translocation breakpoint, STEAP, OFA/iLRP, Chondroitin sulfate proteoglycan 4 (CSPG4), Epithelial tumor antigen, alphafetoprotein, CD19, CA-125, and a combination thereof.

27. The method of any one of claims 1-26, wherein the immunogenic composition is capable of expanding immune cells from the individual or a donor with tumor-targeting ability.

28. The method of any one of claims 1-27, wherein the immunogenic composition comprises at least one antigen present on the cancer afflicting the individual or at least one molecule similar to an antigen present on the cancer afflicting the individual.

29. The method of claim 28, wherein the antigen(s) or molecule(s) comprise at least one antigen or molecule that is derived from a tumor that is histologically similar to the cancer afflicting the individual.

30. The method of claim 29, wherein the antigen or molecule is derived from the histologically-similar tumor by lysis, mRNA extraction, exosome extraction, or a combination thereof.

31. The method of claim 28, wherein at least one of the antigens present on the cancer afflicting the individual comprises a tumor associated protein.

32. The method of claim 31, wherein the tumor associated protein is selected from the group consisting of Fos-related antigen 1, LCK, FAP, VEGFR2, NA17, PDGFR-beta, PAP, MAD-CT-2, Tie-2, PSA, protamine 2, legumain, endosialin, prostate stem cell antigen, carbonic anhydrase IX, STn, Page4, proteinase 3, GM3 ganglioside, tyrosinase, MART1, gp100, SART3, RGS5, SSX2, Globol1, Tn, CEA, hCG, PRAME, XAGE-1, AKAP-4, TRP-2, B7H3, sperm fibrous sheath protein, CYP1B1, HMWMAA, sLe(a), MAGE A1, GD2, PSMA, mesothelin, fucosyl GM1, GD3, sperm protein 17, NY-ESO-1, PAX5, AFP, polysialic acid, EpCAM, MAGE-A3, mutant p53, ras, mutant ras, NY-BR1, PAX3, HER2/neu, OY-TES1, HPV E6 E7, PLAC1, hTERT, BORIS, ML-IAP, idiotype of b cell lymphoma or multiple myeloma, EphA2, EGFRvIII, cyclin B1, RhoC, androgen receptor, surviving, MYCN, wildtype p53, LMP2, ETV6-AML, MUC1, BCR-ABL, ALK, WT1, ERG (TMPRSS2 ETS fusion gene), sarcoma translocation breakpoint, STEAP, OFA/iLRP, Chondroitin sulfate proteoglycan 4 (CSPG4), Epithelial tumor antigen, alphafetoprotein, CD19, CA-125, and a combination thereof.

33. The method of any one of claims 1-32, wherein the immunogenic composition comprises a peptide, plurality of peptides, and/or an altered peptide ligand derived from a protein selected from the group consisting of Fos-related antigen 1, LCK, FAP, VEGFR2, NA17, PDGFR-beta, PAP, MAD-CT-2, Tie-2, PSA, protamine 2, legumain, endosialin, prostate stem cell antigen, carbonic anhydrase IX, STn, Page4, proteinase 3, GM3 ganglioside, tyrosinase, MART1, gp100, SART3, RGS5, SSX2, Globol1, Tn, CEA, hCG, PRAME, XAGE-1, AKAP-4, TRP-2, B7H3, sperm fibrous sheath protein, CYP1B1, HMWMAA, sLe(a), MAGE A1, GD2, PSMA, mesothelin, fucosyl GM1, GD3, sperm protein 17, NY-ESO-1, PAX5, AFP, polysialic acid, EpCAM, MAGE-A3, mutant p53, ras, mutant ras, NY-BR1, PAX3, HER2/neu, OY-TES1, HPV E6 E7, PLAC1, hTERT, BORIS, ML-IAP, idiotype of b cell lymphoma or multiple myeloma, EphA2, EGFRvIII, cyclin B1, RhoC, androgen receptor, surviving, MYCN, wildtype p53, LMP2, ETV6-AML, MUC1, BCR-ABL, ALK, WT1, ERG (TMPRSS2 ETS fusion gene), sarcoma translocation breakpoint, STEAP, OFA/iLRP, Chondroitin sulfate proteoglycan 4 (CSPG4), Epithelial tumor antigen, alphafetoprotein, CD19, CA-125, and a combination thereof.

34. The method of any one of claims 1-33, wherein the immunogenic composition is matched with the HLA haplotype of the individual.

35. The method of any one of claims 1-34, wherein the immunogenic composition is given with one or more adjuvants.

36. The method of claim 35, wherein the adjuvant comprises a composition capable of augmenting antigen presentation.

37. The method of claim 36, wherein the stimulator of antigen presentation comprises a toll like receptor (TLR).

38. The method of claim 37, wherein the stimulator of antigen presentation is TLR-2.

39. The method of claim 38, wherein TLR-2 is activated by one or more compounds selected from the group consisting of Pam3cys4, Heat Killed Listeria monocytogenes (HKLM), FSL-1, and a combination thereof.

40. The method of claim 37, wherein the stimulator of antigen presentation is TLR-3.

41. The method of claim 40, wherein TLR-3 is activated by Poly IC, double stranded RNA, or both.

42. The method of claim 41, wherein the double stranded RNA comprises double stranded RNA of mammalian origin and/or prokaryotic origin.

43. The method of claim 41, wherein the double stranded RNA is derived from leukocyte extract.

44. The method of claim 43, wherein said leukocyte extract comprises a heterogeneous composition derived from freeze-thawed leukocytes, wherein the freeze-thawed leukocytes are dialyzed for compounds less than 15 kDa.

45. The method of claim 37, wherein the stimulator of antigen presentation is TLR-4.

46. The method of claim 45, wherein TLR-4 is activated by a composition selected from the group consisting of lipopolysaccharide, HMGB-1, a peptide derived from HMGB-1, and a combination thereof.

47. The method of claim 46, wherein the peptide derived from HMGB-1 comprises hp91.

48. The method of claim 45, wherein TLR-4 is activated by a peptide comprising at least 80, 85, 90, 95, 96, 97, 98, 99, or 100 percent identity to the peptide with an amino acid sequence of SEQ ID NO:1.

49. The method of claim 37, wherein the stimulator of antigen presentation is TLR-5.

50. The method of claim 49, wherein TLR-5 is activated by flagellin.

51. The method of claim 37, wherein the stimulator of antigen presentation is TLR-7.

52. The method of claim 51, wherein TLR-7 is activated by imiquimod.

53. The method of claim 37, wherein the stimulator of antigen presentation is TLR-8.

54. The method of claim 53, wherein TLR-4 is activated by resmiquimod.

55. The method of claim 37, wherein the stimulator of antigen presentation is TLR-9.

56. The method of claim 55, wherein TLR-9 is activated by CpG DNA.

57. The method of claim 36, wherein the stimulator of antigen presentation comprises at least one agent capable of upregulating expression of at least one costimulatory molecule on antigen presenting cells.

58. The method of claim 57, wherein at least one of the costimulatory molecules comprise CD40, CD80, CD86, or a combination thereof.

59. The method of claim 57, wherein at least one of the agents capable of upregulating expression of costimulatory molecules comprise an activator of NF-kappa-B.

60. The method of claim 59, wherein the activator of NF-kappa-B comprises an inhibitor of i-kappa-B, an activator of one or more PAMP receptors, or both.

61. The method of claim 60, wherein the PAMP receptor is selected from the group consisting of MDA5, RIG-1, NOD, and a combination thereof.

62. The method of claim 57, wherein at least one of the agents capable of upregulating expression of costimulatory molecules comprise an activator of the JAK-STAT pathway.

63. The method of claim 62, wherein the activator of the JAK-STAT pathway comprises interferon gamma.

64. The method of any one of claims 1-63, wherein the composition capable of augmenting antigen presentation locally comprises dendritic cells.

65. The method of claim 64, wherein said dendritic cell is activated with a TLR agonist, a PAMP agonist, in vivo administration of GM-CSF, in vivo administration of FLT-3L, or a combination thereof.

66. The method of either claim 64 or claim 65, wherein said dendritic cell is generated from monocytes.

67. The method of any one of claims 64-66, wherein said dendritic cell is autologous to the individual in need of therapy.

68. The method of any one of claims 64-66, wherein said dendritic cell is allogenic to the individual in need of therapy.

69. The method of any one of claims 1-68, wherein inducing cell death comprises administration of localized radiation therapy.

70. The method of any one of claims 1-69, wherein inducing cell death comprises cryoablation.

71. The method of any one of claims 1-70, wherein inducing cell death comprises localized administration of hyperthermia.

72. The method of any one of claims 1-70, wherein inducing cell death comprises localized administration of chemotherapy.

73. The method of claim 82, wherein chemotherapy is selected from the group consisting of acivicin, aclarubicin, acodazole hydrochloride, acronine, adozelesin, aldesleukin, altretamine, ambomycin, ametantrone acetate, aminoglutethimide, amsacrine, anastrozole, anthramycin, asparaginase, asperlin, azacitidine, azetepa, azotomycin, batimastat, benzodepa, bicalutamide, bisantrene hydrochloride, bisnafide dimesylate, bizelesin, bleomycin sulfate, brequinar sodium, bropirimine, busulfan, cactinomycin, calusterone, caracemide, carbetimer, carboplatin, carmustine, carubicin hydrochloride, carzelesin, cedefingol, chlorambucil, cirolemycin, cisplatin, cladribine, crisnatol mesylate, cyclophosphamide, cytarabine, dacarbazine, dactinomycin, daunorubicin hydrochloride, decitabine, dexormaplatin, dezaguanine, dezaguanine mesylate, diaziquone, docetaxel, doxorubicin, doxorubicin hydrochloride, droloxifene, droloxifene citrate, dromostanolone propionate, duazomycin, edatrexate, eflornithine hydrochloride, elsamitrucin, enloplatin, enpromate, epipropidine, epirubicin hydrochloride, erbulozole, esorubicin hydrochloride, estramustine, estramustine phosphate sodium, etanidazole, etoposide, etoposide phosphate, etoprine, fadrozole hydrochloride, fazarabine, fenretinide, floxuridine, fludarabine phosphate, fluorouracil, fluorocitabine, fosquidone, fostriecin sodium, gemcitabine, gemcitabine hydrochloride, hydroxyurea, idarubicin hydrochloride, ifosfamide, ilmofosine, interleukin II (including recombinant interleukin II, or rIL2), interferon alfa-2a, interferon alfa-2b, interferon alfa-n1, interferon alfa-n3, interferon beta-I a, interferon gamma-I b, iproplatin, irinotecan hydrochloride, lanreotide acetate, letrozole, leuprolide acetate, liarozole hydrochloride, lometrexol sodium, lomustine, losoxantrone hydrochloride, masoprocol, maytansine, mechlorethamine hydrochloride, megestrol acetate, melengestrol acetate, melphalan, menogaril, mercaptopurine, methotrexate, methotrexate sodium, metoprine, meturedepa, mitindomide, mitocarcin, mitocromin, mitomalcin, mitomycin, mitosper, mitotane, mitoxantrone hydrochloride, mycophenolic acid, nocodazole, nogalamycin, ormaplatin, oxisuran, paclitaxel, pegaspargase, peliomycin, pentamustine, peplomycin sulfate, perfosfamide, pipobroman, piposulfan, piroxantrone hydrochloride, plicamycin, plomestane, porfimer sodium, porfiromycin, prednimustine, procarbazine hydrochloride, puromycin, puromycin hydrochloride, pyrazofurin, riboprine, rogletimide, safingol, safingol hydrochloride, semustine, simtrazene, sparfosate sodium, sparsomycin, spirogermanium hydrochloride, spiromustine, spiroplatin, streptonigrin, streptozocin, sulofenur, talisomycin, tecogalan sodium, tegafur, teloxantrone hydrochloride, temoporfin, teniposide, teroxirone, testolactone, thiamiprine, thioguanine, thiotepa, tiazofurin, tirapazamine, toremifene citrate, trestolone acetate, triciribine phosphate, trimetrexate, trimetrexate glucuronate, triptorelin, tubulozole hydrochloride, uracil mustard, uredepa, vapreotide, verteporfin, vinblastine sulfate, vincristine sulfate, vindesine, vindesine sulfate, vinepidine sulfate, vinglycinate sulfate, vinleurosine sulfate, vinorelbine tartrate, vinrosidine sulfate, vinzolidine sulfate, vorozole, zeniplatin, zinostatin, zorubicin hydrochloride, and a combination thereof.

74. The method of any one of claims 1-73, wherein prior to the steps of a)-d), a state of lymphopenia is induced in the individual.

75. The method of claim 74, wherein said lymphopenia is sufficient to induce homeostatic expansion of lymphocytes in the individual.

76. The method of claim 74, wherein said lymphopenia is sufficient to induce homeostatic proliferation of lymphocytes endogenous to the individual.

77. The method of claim 76, wherein said homeostatic expansion allows for an over 50% reduction in need of said lymphocytes for costimulatory signals.

78. The method of claim 74, wherein said lymphopenia is achieved by irradiation, administration of cyclophosphamide, or both.

79. The method of claim 78, wherein said irradiation is total lymphoid irradiation.

80. The method of any one of claim 1-79, wherein increased propensity of lymphocytes for activation is induced by treatment with one or more lymphocyte mitogens.

81. The method of claim 80, wherein said lymphocyte mitogen comprises IL-2 treatment.

82. The method of claim 80, wherein said lymphocyte mitogen comprises IL-7 treatment.

83. The method of claim 80, wherein said lymphocyte mitogen comprises IL-15 treatment.

84. The method of any one of claims 1-83, wherein one or more immune de-repressing agents are administered to the individual, wherein said agent comprises one or more phosphodiesterase (PDE)-5 inhibitors.

85. The method of claim 84, wherein said PDE-5 inhibitor is selected from the group consisting of of: Acetildenafi, Aildenafil, Avanafil, Benzamidenafil, Homosildenafil, Icariin, Lodenafil, Mirodenafil, Nitrosoprodenafil, Sildenafil, Sulfoaildenafil, Tadalafil, Udenafil, Vardenafil, Zaprinast, and a combination thereof.

86. The method of any one of claims 1-85, wherein the individual has a brain tumor.

87. The method of claim 86, wherein said brain tumor is selected from the group consisting of a glioblastoma, a glioblastoma multiforme, an oligodendroglioma, a primitive neuroectodermal tumor, an astrocytoma, an ependymoma, an oligodendroglioma, a medulloblastoma, a meningioma, a pituitary carcinoma, a neuroblastoma, a craniopharyngioma, and a combination thereof.


SEQUENCE LISTING (1 sequence)

SEQ ID NO: 1 - protein, 68 aa, Unknown [sequence omitted]